(19) United States
(12) Patent Application Publication
(10) Pub. No.: US 2022/0033800 A1
(43) Pub. Date: Feb. 3, 2022

(54) ENGINEERED BIOSYNTHETIC PATHWAYS FOR PRODUCTION OF 1,5-DIAMINOPENTANE BY FERMENTATION

(71) Applicant: Zymergen Inc., Emeryville, CA (US)

(72) Inventors: Aaron Miller, Emeryville, CA (US); Zhihao Wang, Emeryville, CA (US); Jeffrey Mellin, Emeryville, CA (US); Murtaza Shabbir Hussain, Emeryville, CA (US); Steven M. Edgar, Albany, CA (US)

(73) Assignee: Zymergen Inc., Emeryville, CA (US)

(21) Appl. No.: 17/297,383

(22) PCT Filed: Nov. 21, 2019
(86) PCT No.: PCT/US2019/062664

§ 371 Date: May 26, 2021

Related U.S. Application Data
(60) Provisional application No. 62/774,016, filed on Nov. 30, 2018.

(51) Int. Cl. C12N 9/88; C12P 13/00

(52) U.S. Cl. CPC C12N 9/88; C12P 13/001; C12Y 401/01018

(57) ABSTRACT

The present disclosure describes the engineering of microbial cells for fermentative production of 1,5-diaminopentane and provides novel engineered microbial cells and cultures, as well as related 1,5-diaminopentane production methods.

### CROSS-REFERENCE TO RELATED APPLICATIONS

This application claims priority to and benefit of U.S. provisional application No. 62/774,016, filed on Nov. 30, 2018, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Nov. 20, 2019, is named ZMGNP026WO_SL.txt. and is 1,590,352 bytes in size.

### FIELD OF THE DISCLOSURE

The present disclosure relates generally to the area of engineering microbes for production of 1,5-diaminopentane by fermentation.

### BACKGROUND

1,5-diaminopentane is a metabolite in the degradation pathway of lysine. Specifically, 1,5-diaminopentane is produced by decarboxylation of lysine.

In zebrafish, the trace amine-associated receptor 13c (or TAAR13c) has been identified as a high-affinity receptor for cadaverin.[5] In humans, molecular modelling and docking experiments have shown that cadaverine fits into the binding pocket of the human TAAR6 and TAAR8.

1,5-diaminopentane is a chemical precursor to pentolinium, which is a ganglionic blocking agent that acts by inhibiting the nicotinic acetylcholine receptor.

### SUMMARY

The disclosure provides engineered microbial cells, cultures of the microbial cells, and methods for the production of 1,5-diaminopentane, including the following:

Embodiment 1: An engineered microbial cell that expresses a non-native lysine decarboxylase, wherein the engineered microbial cell produces 1,5-diaminopentane.

Embodiment 2: The engineered microbial cell of embodiment 1, wherein the engineered microbial cell also expresses a non-native 1,5-diaminopentane transporter.

Embodiment 3: The engineered microbial cell of embodiment 1 or embodiment 2, wherein the engineered microbial cell expresses one or more additional enzyme(s) selected from an additional non-native lysine decarboxylase and/or an additional non-native 1,5-diaminopentane transporter.

Embodiment 4: The engineered microbial cell of embodiment 3, wherein the additional enzyme(s) are from a different organism than the corresponding enzyme in embodiment 1 or embodiment 2.

Embodiment 5: The engineered microbial cell of embodiment 3 or embodiment 4, wherein the additional enzyme(s) comprise(s) one or more additional copies of the corresponding enzyme in embodiment 1 or embodiment 2.

Embodiment 6: The engineered microbial cell of any of embodiments 1-5, wherein the engineered microbial cell includes increased activity of one or more upstream lysine pathway enzyme(s), said increased activity being increased relative to a control cell.

Embodiment 7: The engineered microbial cell of any of embodiments 1-6, wherein the engineered microbial cell includes increased activity of one or more enzyme(s) that increase the supply of the reduced form of nicotinamide adenine dinucleotide phosphate (NADPH), said increased activity being increased relative to a control cell.

Embodiment 8: The engineered microbial cell of embodiment 7, wherein the one or more enzyme(s) that increase the supply of the reduced form of NADPH is selected from the group consisting of pentose phosphate pathway enzymes, NADP+-dependent glyceraldehyde 3-phosphate dehydrogenase (GAPDH), and NADP+-dependent glutamate dehydrogenase.

Embodiment 9: The engineered microbial cell of any one of embodiments 1-8, wherein the engineered microbial cell includes reduced activity of one or more enzyme(s) that consume one or more lysine pathway precursors, said reduced activity being reduced relative to a control cell.

Embodiment 10: The engineered microbial cell of any one of embodiments 1-9, wherein the engineered microbial cell includes reduced activity of a native lysine exporter, said reduced activity being reduced relative to a control cell.

Embodiment 11: The engineered microbial cell of embodiment 10, wherein the native lysine exporter is Corynebacterium glutamicum lysE or an ortholog thereof.

Embodiment 12: The engineered microbial cell of any one of embodiments 1-11, wherein the engineered microbial cell includes reduced expression of the C. glutamicum NCg10561 gene or an ortholog thereof, said reduced expression being reduced relative to a control cell.

Embodiment 13: The engineered microbial cell of any one of embodiments 1-12, wherein the engineered microbial cell includes reduced expression of the C. glutamicum trpB gene or an ortholog thereof, said reduced expression being reduced relative to a control cell.

Embodiment 14: The engineered microbial cell of any one of embodiments 9-13, wherein the reduced activity is achieved by one or more means selected from the group consisting of gene deletion, gene disruption, altering regulation of a gene, and replacing a native promoter with a less active promoter.

Embodiment 15: An engineered microbial cell, wherein the engineered microbial cell includes means for expressing a non-native lysine decarboxylase, and wherein the engineered microbial cell produces 1,5-diaminopentane.

Embodiment 16: The engineered microbial cell of embodiment 15, wherein the engineered microbial cell also includes means for expressing a non-native 1,5-diaminopentane transporter.

Embodiment 17: The engineered microbial cell of embodiment 15 or embodiment 16, wherein the engineered microbial cell means for expressing one or more additional enzyme(s) selected from an additional non-native lysine decarboxylase and/or an additional non-native 1,5-diaminopentane transporter.

Embodiment 18: The engineered microbial cell of embodiment 17, wherein the additional enzyme(s) are from a different organism than the corresponding enzyme in embodiment 15 or embodiment 16.

Embodiment 19: The engineered microbial cell of any of embodiments 15-18 wherein the engineered microbial cell includes means for increasing activity of one or more upstream lysine pathway enzyme(s), said activity being increased relative to a control cell.

Embodiment 20: The engineered microbial cell of any of embodiments 15-19, wherein the engineered microbial cell includes means for increasing activity of one or more enzyme(s) that increase the NADPH supply, said activity being increased relative to a control cell.

Embodiment 21: The engineered microbial cell of embodiment 20, wherein the one or more enzyme(s) that increase the supply of the reduced form of nicotinamide adenine dinucleotide phosphate (NADPH) is selected from the group consisting of pentose phosphate pathway enzymes, NADP+-dependent glyceraldehyde 3-phosphate dehydrogenase (GAPDH), and NADP+-dependent glutamate dehydrogenase.

Embodiment 22: The engineered microbial cell of any one of embodiments 15-21, wherein the engineered microbial cell includes means for reducing activity of one or more enzyme(s) that consume one or more lysine pathway precursors, said activity being reduced relative to a control cell.

Embodiment 23: The engineered microbial cell of any one of embodiments 15-22, wherein the engineered microbial cell includes means for reducing activity of a native lysine exporter, said activity being reduced relative to a control cell.

Embodiment 24: The engineered microbial cell of embodiment 23, wherein the native lysine exporter is Corynebacterium glutamicum lysE or an ortholog thereof.

Embodiment 25: The engineered microbial cell of any one of embodiments 15-24, wherein the engineered microbial cell includes means for reducing expression of the C. glutamicum NCg10561 gene or an ortholog thereof, said expression being reduced relative to a control cell.

Embodiment 26: The engineered microbial cell of any one of embodiments 15-25, wherein the engineered microbial cell includes means for reducing expression of the C. glutamicum trpB gene or an ortholog thereof, said expression being reduced relative to a control cell.

Embodiment 27: The engineered microbial cell of any one of embodiments 1-26, wherein the engineered microbial cell is a bacterial cell.

Embodiment 28: The engineered microbial cell of embodiment 27, wherein the bacterial cell is a cell of the genus Corynebacteria.

Embodiment 29: The engineered microbial cell of embodiment 28, wherein the bacterial cell is a cell of the species glutamicum.

Embodiment 30: The engineered microbial cell of embodiment 29, wherein the non-native lysine decarboxylase includes a lysine decarboxylase having at least 70% amino acid sequence identity with a lysine decarboxylase selected from the group consisting of Escherichia coli, Vibrio cholerae, Candidatus Burkholderia crenata, butyrate-producing bacterium, and any combination thereof.

Embodiment 31: The engineered microbial cell of embodiment 30, wherein the cell includes at least three different lysine decarboxylases.

Embodiment 32: The engineered microbial cell of embodiment 31, wherein the engineered microbial cell includes three non-native lysine decarboxylases having at least 70% amino acid sequence identity with each of the lysine decarboxylases from Escherichia coli, Candidatus Burkholderia crenata, and butyrate-producing bacterium.

Embodiment 33: The engineered microbial cell of embodiment 32, wherein the engineered microbial cell additionally includes a non-native lysine decarboxylase having at least 70% amino acid sequence identity with a lysine decarboxylase from a mine drainage metagenome.

Embodiment 34: The engineered microbial cell of embodiment 33, wherein the lysine decarboxylases from Escherichia coli, Candidatus Burkholderia crenata, butyrate-producing bacterium, and the mine drainage metagenome comprise SEQ ID NOs:87, 97, 30, and 93.

Embodiment 35: The engineered microbial cell of embodiment 27, wherein the bacterial cell is a cell of the genus Bacillus.

Embodiment 36: The engineered microbial cell of embodiment 35, wherein the bacterial cell is a cell of the species subtilis.

Embodiment 37: The engineered microbial cell of embodiment 36, wherein the non-native lysine decarboxylase includes a lysine decarboxylase having at least 70% amino acid sequence identity with a lysine decarboxylase selected from the group consisting of a Clostridium species, Staphylococcus aureus, and any combination thereof.

Embodiment 38: The engineered microbial cell of embodiment 37, wherein the cell includes at least three different lysine decarboxylases.

Embodiment 39: The engineered microbial cell of embodiment 38, wherein the engineered microbial cell includes three non-native lysine decarboxylases having at least 70% amino acid sequence identity with each of the lysine decarboxylases from Clostridium CAG:221, Clostridium CAG:288, and Staphylococcus aureus.

Embodiment 40: The engineered microbial cell of any one of embodiments 1-26, wherein the engineered microbial cell includes a fungal cell.

Embodiment 41: The engineered microbial cell of embodiment 40, wherein the engineered microbial cell includes a yeast cell.

Embodiment 42: The engineered microbial cell of embodiment 41, wherein the yeast cell is a cell of the genus Saccharomyces.

Embodiment 43: The engineered microbial cell of embodiment 42, wherein the yeast cell is a cell of the species cerevisiae.

Embodiment 44: The engineered microbial cell of any one of embodiments 1-43, wherein the non-native lysine decarboxylase includes a lysine decarboxylase having at least 70% amino acid sequence identity with a lysine decarboxylase selected from the group consisting of Yersinia enterocolitica, Castellaniella detragans, Prochorococcus marinus, and any combination thereof.

Embodiment 45: The engineered microbial cell of embodiment 44, wherein the cell includes at least three different lysine decarboxylases.

Embodiment 46: The engineered microbial cell of embodiment 45, wherein the engineered microbial cell includes three non-native lysine decarboxylases having at least 70% amino acid sequence identity with each of the lysine decarboxylases from Yersinia enterocolitica, Castellaniella detragans, and Prochorococcus marinus.

Embodiment 47: The engineered microbial cell of any one of embodiments 1-46, wherein, when cultured, the engineered microbial cell produces 1,5-diaminopentane at a level at least 5 mg/L of culture medium.

Embodiment 48: The engineered microbial cell of embodiment 47, wherein, when cultured, the engineered microbial cell produces 1,5-diaminopentane at a level at least 5 gm/L of culture medium.

Embodiment 49: The engineered microbial cell of embodiment 48, wherein, when cultured, the engineered microbial cell produces 1,5-diaminopentane at a level at least 25 gm/L of culture medium.

Embodiment 50: A method of culturing engineered microbial cells according to any one of embodiments 1-49, the method including culturing the cells under conditions suitable for producing 1,5-diaminopentane.

Embodiment 51: The method of embodiment 50, wherein the method includes fed-batch culture, with an initial glucose level in the range of 1-100 g/L, followed controlled sugar feeding.

Embodiment 52: The method of embodiment 50 or embodiment 51, wherein the fermentation substrate includes glucose and a nitrogen source selected from the group consisting of urea, an ammonium salt, ammonia, and any combination thereof.

Embodiment 53: The method of any one of embodiments 50-52, wherein the culture is pH-controlled during culturing.

Embodiment 54: The method of any one of embodiments 50-53, wherein the culture is aerated during culturing.

Embodiment 55: The method of any one of embodiments 50-54, wherein the engineered microbial cells produce 1,5-diaminopentane at a level at least 5 mg/L of culture medium.

Embodiment 56: The method of any one of embodiments 50-55, wherein the method additionally includes recovering 1,5-diaminopentane from the culture.

Embodiment 57: A method for preparing 1,5-diaminopentane using microbial cells engineered to produce 1,5-diaminopentane, the method including: (a) expressing a non-native lysine decarboxylase in microbial cells; (b) cultivating the microbial cells in a suitable culture medium under conditions that permit the microbial cells to produce 1,5-diaminopentane, wherein the 1,5-diaminopentane is released into the culture medium; and (c) isolating 1,5-diaminopentane from the culture medium.

### BRIEF DESCRIPTION OF THE DRAWINGS

FIG. 1: Biosynthetic pathway for 1,5-diaminopentane.

FIG. 2: 1,5-diaminopentane titers measured in the extracellular broth following fermentation by first-round engineered host Corynebacteria glutamicum. (See also Example 1.)

FIG. 3: 1,5-diaminopentane titers measured in the extracellular broth following fermentation by first-round engineered host Saccharomyces cerevisiae. (See also Example 1.)

FIG. 4: 1,5-diaminopentane titers measured in the extracellular broth following fermentation by first-round engineered host Bacillus subtilis. (See also Example 1.)

FIG. 5: 1,5-diaminopentane titers measured in the extracellular broth following fermentation by second-round engineered host Corynebacteria glutamicum. (See also Example 1.)

FIG. 6: 1,5-diaminopentane titers measured in the extracellular broth following fermentation by Corynebacteria glutamicum engineered to delete the NCg10561 gene (NCg10561_del) or delete the NCg12931 gene, which encodes the beta subunit of tryptophan synthase (NCg12931_P3221).

FIG. 7: Integration of Promoter-Gene-Terminator into Saccharomyces cerevisiae and Yarrowia lipolytica.

FIG. 8: Promoter replacement in Saccharomyces cerevisiae and Yarrowia lipolytica.

FIG. 9: Targeted gene deletion in Saccharomyces cerevisiae and Yarrowia lipolytica.

FIG. 10: Integration of Promoter-Gene-Terminator into Corynebacteria glutamicum and Bacillus subtilis.

FIG. 11: 1,5-diaminopentane titers measured in the extracellular broth following fermentation by third-round engineered host Corynebacteria glutamicum. (See also Example 1.)

FIG. 12: Bioreactor production runs of engineered Corynebacteria glutamicum strain CgCADAV_107 resulted a 1,5-diaminopentane titer of 27 g/L. (See Example 2.)

### DETAILED DESCRIPTION

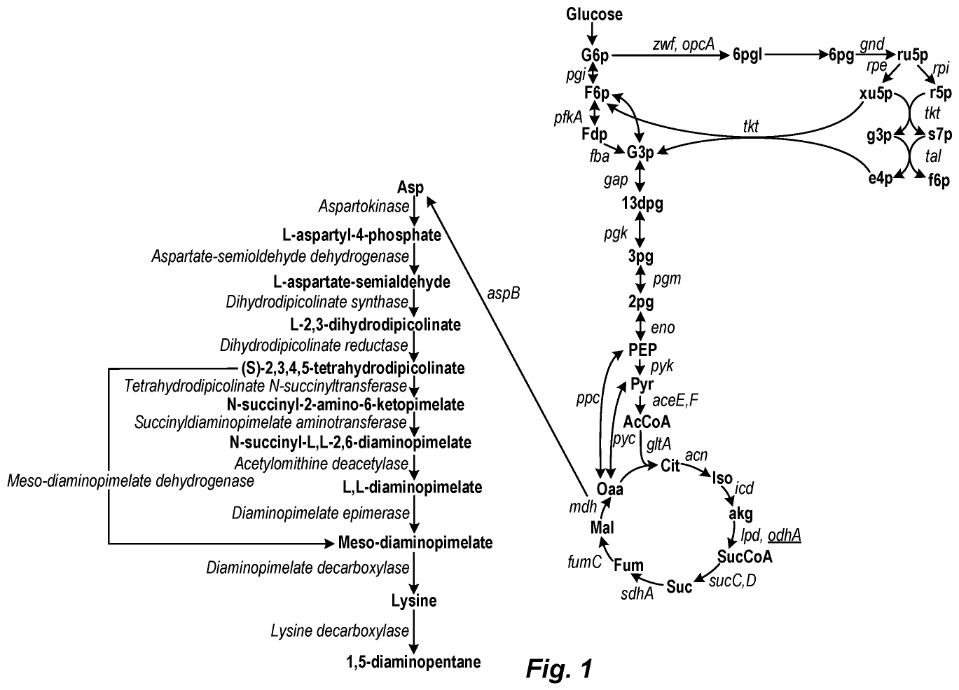

This disclosure describes a method for the production of the small molecule 1,5-diaminopentane via fermentation by a microbial host from simple carbon and nitrogen sources, such as glucose and urea, respectively. This objective can be achieved by introducing a non-native metabolic pathway into a suitable microbial host for industrial fermentation of chemical products. Illustrative hosts include Saccharomyces cerevisiae, Yarrowia lypolytica, Corynebacteria glutamicum, and Bacillus subtilis. The engineered metabolic pathway links the central metabolism of the host to a non-native pathway to enable the production of 1,5-diaminopentane. The simplest embodiment of this approach is the expression of an enzyme, such as a non-native lysine decarboxylase enzyme, in a microbial host strain that has the other enzymes necessary for 1,5-diaminopentane production (see FIG. 1; i.e., any strain that produces lysine), which is true of all of the illustrative hosts noted above.

The following disclosure describes how to engineer a microbe with the necessary characteristics to produce industrially feasible titers of 1,5-diaminopentane from simple carbon and nitrogen sources. Active lysine decarboxylases have been identified that enable C. glutamicum, S. cerevisiae, and B. subtilis to produce 1,5-diaminopentane, and it has been found that the expression of an additional copy of lysine decarboxylase improves the 1,5-diaminopentane titers. For example, in the work described herein, titers of about 27 gm/L 1,5-diaminopentane in C. glutamicum, about 5 mg/L 1,5-diaminopentane in S. cerevisiae, and about 47 mg/L 1,5-diaminopentane in B. subtilis were achieved.

### Definitions

Terms used in the claims and specification are defined as set forth below unless otherwise specified.

The term “fermentation” is used herein to refer to a process whereby a microbial cell converts one or more substrate(s) into a desired product (such as 1,5-diaminopentane) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell, but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “1,5-diaminopentane” refers to a chemical compound of the formula C₅H₁₄N₂ also known as “pentane-1,5-diamine” and “cadaverine” (CAS#CAS 462-94-2).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., 1,5-diaminopentane) produced by a culture of microbial cells divided by the culture volume.

As used herein with respect to recovering 1,5-diaminopentane from a cell culture, “recovering” refers to separating the 1,5-diaminopentane from at least one other component of the cell culture medium.

Engineering Microbes for 1,5-Diaminopentane Production

1,5-Diaminopentane Biosynthesis Pathway

1,5-diaminopentane is typically derived from lysine in one enzymatic step, requiring the enzyme lysine decarboxylase. The 1,5-diaminopentane biosynthesis pathway is shown in FIG. 1. This enzyme is not expressed naturally in Corynebacteria glutamicum, Saccharomyces cerevisiae, or Bacillus subtilis. 1,5-diaminopentane production is enabled in each of these hosts by the addition of at least one non-native lysine decarboxylase.

Engineering for Microbial 1,5-Diaminopentane Production

Any lysine decarboxylase that is active in the microbial cell being engineered may be introduced into the cell, typically by introducing and expressing the gene(s) encoding the enzyme(s)s using standard genetic engineering techniques. Suitable lysine decarboxylases may be derived from any source, including plant, archaeal, fungal, gram-positive bacterial, and gram-negative bacterial sources. Exemplary sources include, but are not limited to: Escherichia coli, Vibrio cholerae, Candidatus Burkholderia crenata, butyrate-producing bacterium, a Clostridium species (e.g., Clostridium CAG:221, Clostridium CAG:288), Staphylococcus aureus, Yersinia enterocolitica, Castellaniella detragans, and Prochorococcus marinus.

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from an inducible promoter. The heterologous gene(s) can optionally be codon-optimized to enhance expression in the selected microbial host cell.

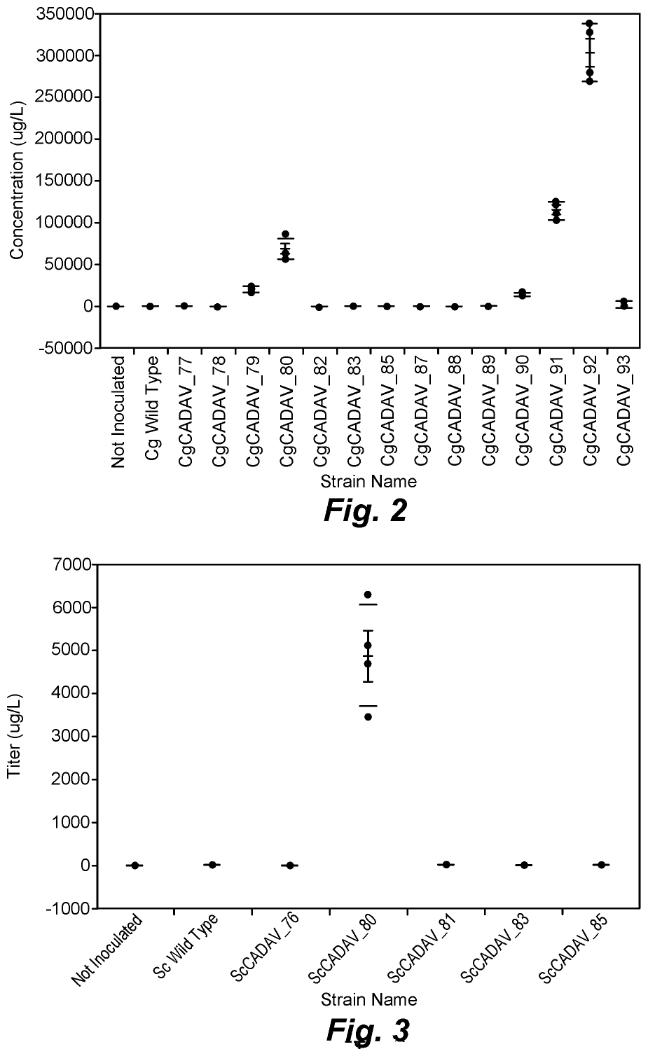

Example 1 shows that, in Corynebacterium glutamicum, an about 300 mg/L titer of 1,5-diaminopentane was achieved in a first round of engineering after integration of the three non-native enzymes. (See FIG. 2.) This strain expressed lysine decarboxylases from of Escherichia coli (strain K12), Escherichia coli O157:H7, and Vibrio cholerae serotype 01 (strain ATCC39315/El Tor Inaba N16961).

Example 1 shows that, in Saccharomyces cerevisiae, a titer of about 5 mg/L was achieved in a first round of engineering after integration of lysine decarboxylases from each of Yersinia enterocolitica W22703, Castellaniella detragans 65Phen, and Prochorococcus marinus str. IT 9314. (See FIG. 3.)

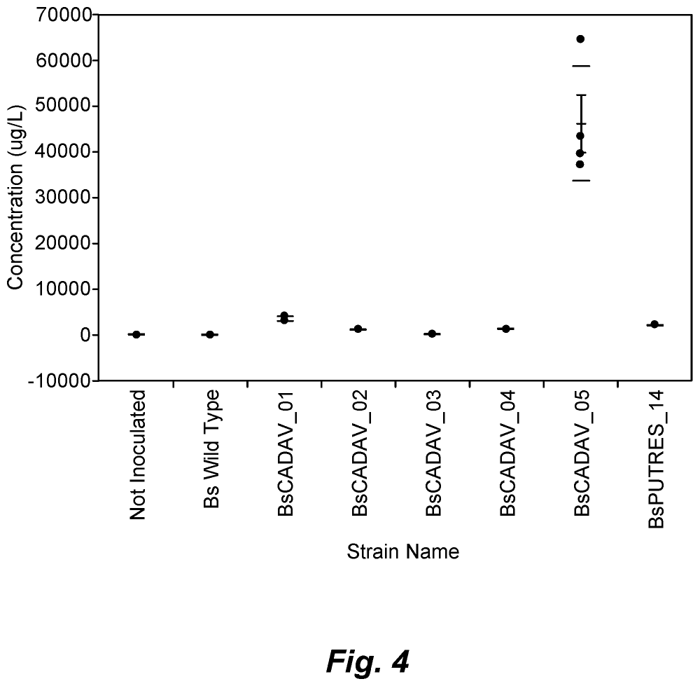

Example 1 shows that, in Bacillus subtilis, a titer of about 47 mg/L was achieved in a first round of engineering after integration of lysine decarboxylases from each of Clostridium CAG:221, Clostridium CAG:288, and Staphylococcus aureus. (See FIG. 4.)

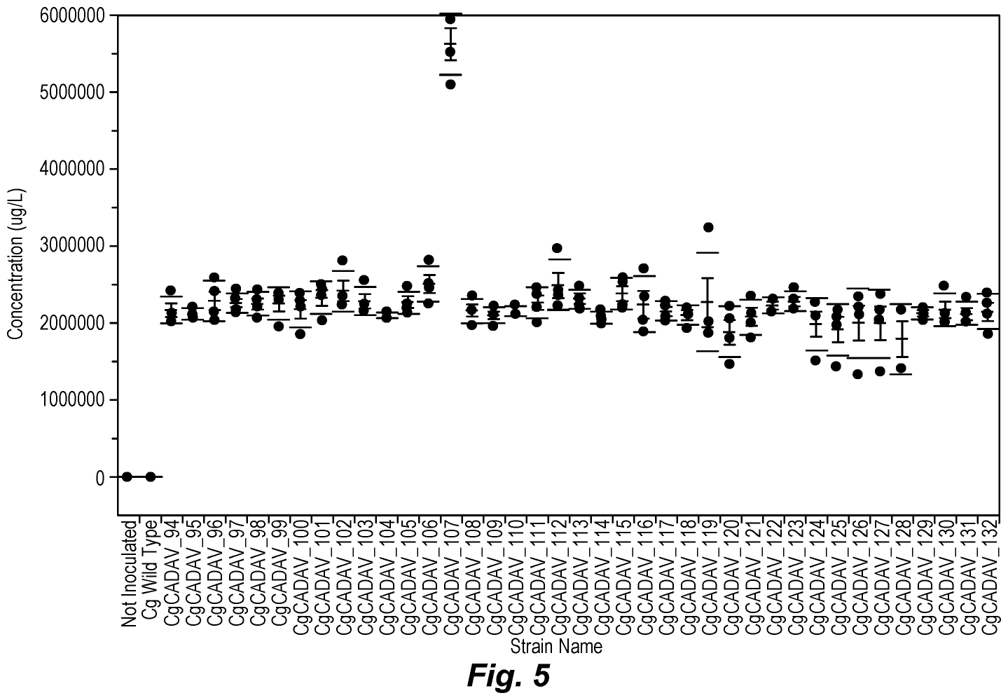

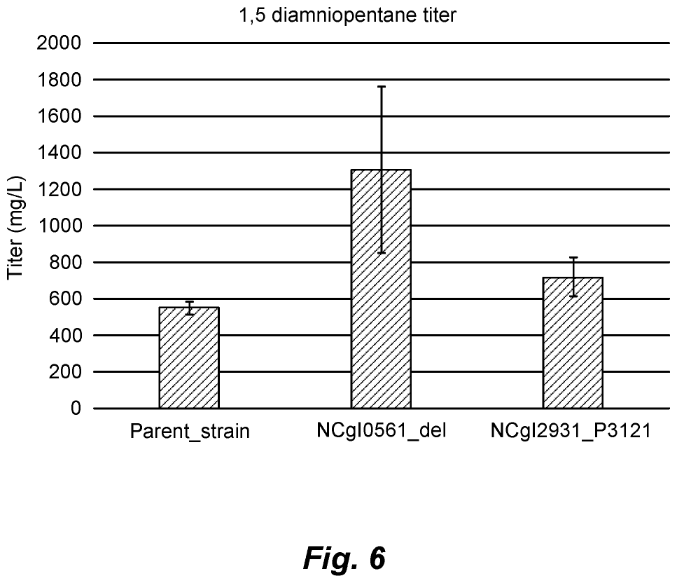

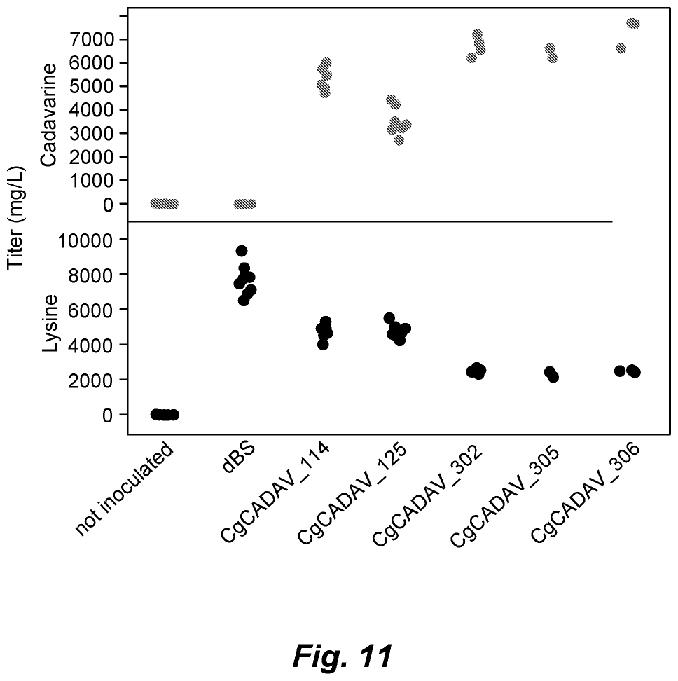

A second round of engineering was carried out in the C. glutamicum (Example 1). A titer of about 5.5 gm/L was achieved after integration of lysine decarboxylases from each of Escherichia coli MS 117-3, Candidatus Burkholderia crenata, and butyrate-producing bacterium SS3/4. (CgCADAV_107; see FIG. 5). A third round of engineering in C. glutamicum (Example 1), that added a lysine decarboxylase from a mine drainage metagenome (SEQ ID NO:93), to these enzymes, increased the titer to 7.0 gm/L (CgCADAV_306; see FIG. 11).

Example 2 shows that a bioreactor production run using CgCADAV_107 (expressing lysine decarboxylases from each of Escherichia coli MS 117-3, Candidatus Burkholderia crenata, and butyrate-producing bacterium SS3/4) achieved a titer of about 27 gm/L 1,5-diaminopentane.

### Increasing the Activity of Upstream Enzymes

One approach to increasing 1,5-diaminopentane production in a microbial cell that is capable of such production is to increase the activity of one or more upstream enzymes in the 1,5-diaminopentane biosynthesis pathway. Upstream pathway enzymes include all enzymes involved in the conversions from a feedstock all the way to into the last native metabolite. Illustrative enzymes, for this purpose, include, but are not limited to, those shown in FIG. 1 in the pathway from aspartate (“Asp”) to lysine. Suitable upstream pathway genes encoding these enzymes may be derived from any available source, including, for example, those discussed above as sources for a lysine decarboxylase.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

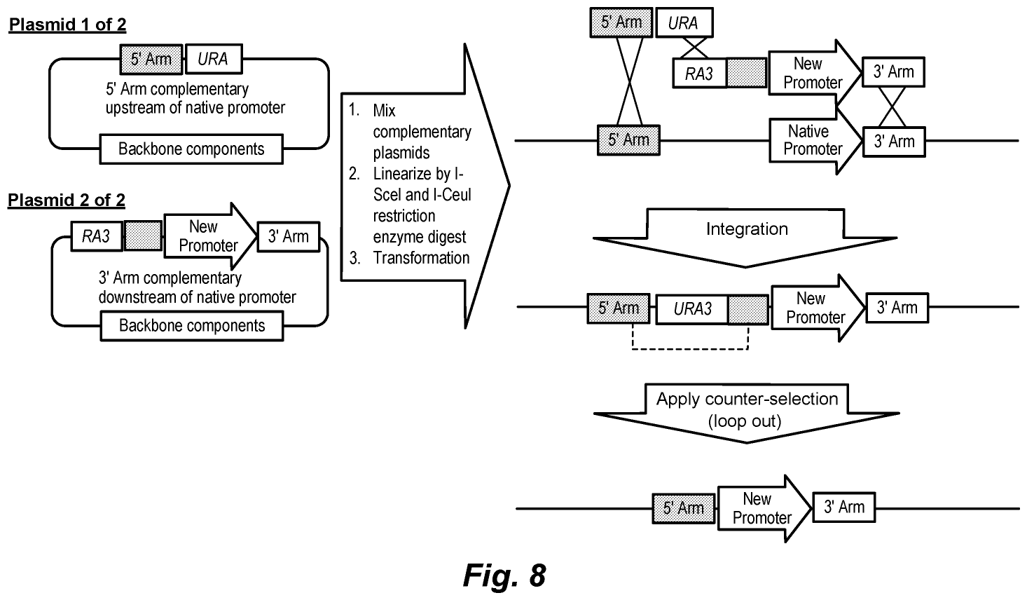

Alternatively, or in addition, one or more promoters can be substituted for native promoters using, for example, a technique such as that illustrated in FIG. 8. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of 1,5-diaminopentane production and expressed from a strong constitutive promoter and/or can optionally be codon-optimized to enhance expression in the selected microbial host cell.

In various embodiments, the engineering of a 1,5-diaminopentane-producing microbial cell to increase the activity of one or more upstream pathway enzymes increases the 1,5-diaminopentane titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 1,5-diaminopentane titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 1,5-diaminopentane titer observed in a 1,5-diaminopentane-producing microbial cell that lacks any increase in activity of upstream pathway enzymes. This reference cell may have one or more other genetic alterations aimed at increasing 1,5-diaminopentane production.

In various embodiments, the 1,5-diaminopentane titers achieved by increasing the activity of one or more upstream pathway enzymes are at least 10, 20, 30, 40, 50, 75, 100, 200, 300, 400, 500, 600, 700, 800, or 900 mg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 gm/L. In various embodiments, the titer is in the range of 10 mg/L to 150 gm/L, 20 mg/L to 140 gm/L, 50 mg/L to 130 gm/L, 100 mg/L to 120 gm/L, 500 mg/L to 110 gm/L or any range bounded by any of the values listed above.

### Increasing the NADPH Supply

Another approach to increasing 1,5-diaminopentane production in a microbial cell that is capable of such production is to increase the supply of the reduced form of nicotinamide adenine dinucleotide phosphate (NADPH), which provides the reducing equivalents for biosynthetic reactions. For example, the activity of one or more enzymes that increase the NADPH supply can be increased by means similar to those described above for upstream pathway enzymes, e.g., by modulating the expression or activity of the native enzyme(s), replacing the native promoter(s) with a stronger and/or constitutive promoter, and/or introducing one or more gene(s) encoding enzymes that increase the NADPH supply. Illustrative enzymes, for this purpose, include, but are not limited to, pentose phosphate pathway enzymes, NADP+-dependent glyceraldehyde 3-phosphate dehydrogenase (GAPDH), and NADP+-dependent glutamate dehydrogenase. Such enzymes may be derived from any available source, including, for example, those discussed above as sources for a lysine decarboxylase.

In various embodiments, the engineering of a 1,5-diaminopentane-producing microbial cell to increase the activity of one or more of such enzymes increases the 1,5-diaminopentane titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 1,5-diaminopentane titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 1,5-diaminopentane titer observed in a 1,5-diaminopentane-producing microbial cell that lacks any increase in activity of such enzymes. This reference cell may have one or more other genetic alterations aimed at increasing 1,5-diaminopentane production.

In various embodiments, the 1,5-diaminopentane titers achieved by increasing the activity of one or more enzymes that increase the NADPH supply are at least 10, 20, 30, 40, 50, 75, 100, 200, 300, 400, 500, 600, 700, 800, or 900 mg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 gm/L. In various embodiments, the titer is in the range of 10 mg/L to 150 gm/L, 20 mg/L to 140 gm/L, 50 mg/L to 130 gm/L, 100 mg/L to 120 gm/L, 500 mg/L to 110 gm/L or any range bounded by any of the values listed above.

### Feedback-Deregulated Enzymes

Since lysine biosynthesis is subject to feedback inhibition, another approach to increasing 1,5-diaminopentane production production in a microbial cell engineered to produce 1,5-diaminopentane production is to introduce feedback-deregulated forms of one or more enzymes that are normally subject to feedback regulation. Examples of such enzymes include glucose-6-phosphate dehydrogenase, ATP phosphoribosyltransferase, and aspartokinase. A feedback-deregulated form can be a heterologous, native enzyme that is less sensitive to feedback inhibition than the native enzyme in the particular microbial host cell. Alternatively, a feedback-deregulated form can be a variant of a native or heterologous enzyme that has one or more mutations or truncations rendering it less sensitive to feedback inhibition than the corresponding native enzyme.

In some embodiments, the feedback-deregulated enzyme need not be “introduced,” in the traditional sense. Rather, the microbial host cell selected for engineering can be one that has a native enzyme that is naturally insensitive to feedback inhibition.

In various embodiments, the engineering of a 1,5-diaminopentane-producing microbial cell to include one or more feedback-regulated enzymes increases the 1,5-diaminopentane titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 1,5-diaminopentane titer is in the range of 10-fold to 1000-fold, 20-fold to 500-fold, 50-fold to 400-fold, 10-fold to 300-fold, or any range bounded by any of the values listed above. These increases are determined relative to the 1,5-diaminopentane titer observed in a 1,5-diaminopentane-producing microbial cell that does not include genetic alterations to reduce feedback regulation. This reference cell may (but need not) have other genetic alterations aimed at increasing 1,5-diaminopentane production, i.e., the cell may have increased activity of an upstream pathway enzyme.

In various embodiments, the 1,5-diaminopentane titers achieved by reducing feedback deregulation are at least 10, 20, 30, 40, 50, 75, 100, 200, 300, 400, 500, 600, 700, 800, or 900 mg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 gm/L. In various embodiments, the titer is in the range of 10 mg/L to 150 gm/L, 20 mg/L to 140 gm/L, 50 mg/L to 130 gm/L, 100 mg/L to 120 gm/L, 500 mg/L to 110 gm/L or any range bounded by any of the values listed above.

### Reduction of Precursor Consumption

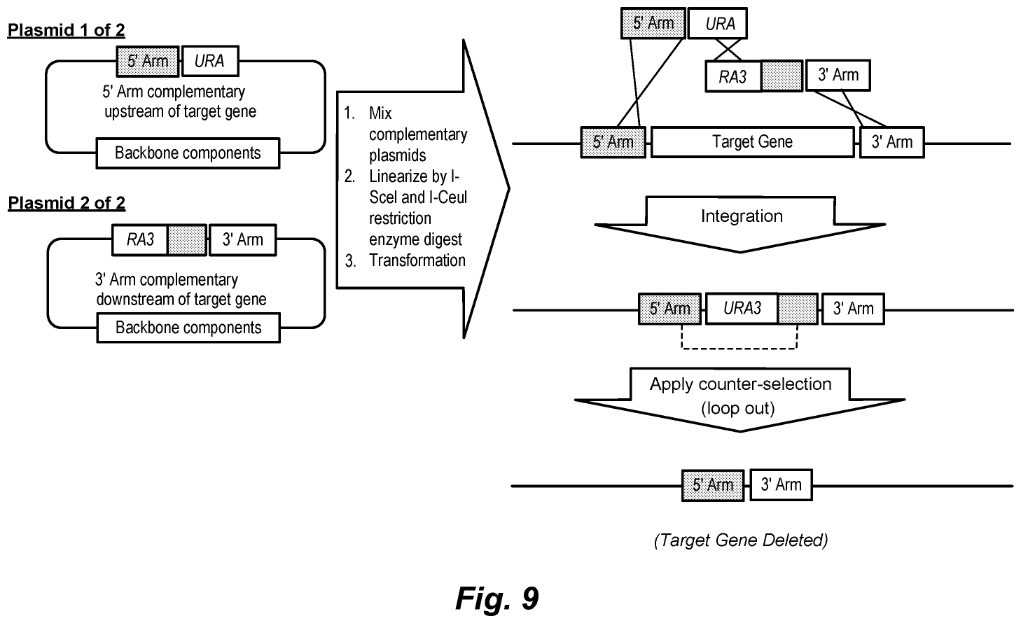

Another approach to increasing 1,5-diaminopentane production in a microbial cell that is capable of such production is to decrease the activity of one or more enzymes that consume one or more 1,5-diaminopentane pathway precursors. In some embodiments, the activity of one or more such enzymes is reduced by modulating the expression or activity of the native enzyme(s). Illustrative enzymes of this type include homoserine dehydrogenase and cell wall biosynthesis pathway genes. The activity of such enzymes can be decreased, for example, by substituting the native promoter of the corresponding gene(s) with a less active or inactive promoter or by deleting the corresponding gene(s). See FIGS. 8 and 9 for examples of schemes for promoter replacement and targeted gene deletion, respectively, in S. cervisiae and Y. lipolytica.

In various embodiments, the engineering of a 1,5-diaminopentane-producing microbial cell to reduce precursor consumption by one or more side pathways increases the 1,5-diaminopentane titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 1,5-diaminopentane titer is in the range of 10-fold to 1000-fold, 20-fold to 500-fold, 50-fold to 400-fold, 10-fold to 300-fold, or any range bounded by any of the values listed above. These increases are determined relative to the 1,5-diaminopentane titer observed in a 1,5-diaminopentane-producing microbial cell that does not include genetic alterations to reduce precursor consumption. This reference cell may (but need not) have other genetic alterations aimed at increasing 1,5-diaminopentane production, i.e., the cell may have increased activity of an upstream pathway enzyme.

In various embodiments, the 1,5-diaminopentane titers achieved by reducing precursor consumption are at least 10, 20, 30, 40, 50, 75, 100, 200, 300, 400, 500, 600, 700, 800, or 900 mg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 gm/L. In various embodiments, the titer is in the range of 10 mg/L to 150 gm/L, 20 mg/L to 140 gm/L, 50 mg/L to 130 gm/L, 100 mg/L to 120 gm/L, 500 mg/L to 110 gm/L or any range bounded by any of the values listed above.

Any of the approaches for increasing 1,5-diaminopentane production described above can be combined, in any combination, to achieve even higher 1,5-diaminopentane production levels.

### Expression of a 1,5-Diaminopentane Transporter

In some embodiments, it is advantageous to recover 1,5-diaminopentane from culture medium. To enhance transport of this compound from inside the engineered microbial cell to the culture medium, a 1,5-diaminopentane transporter that is active in the microbial cell being engineered may be introduced into the cell, typically by introducing and expressing the gene(s) encoding the enzyme(s)s using standard genetic engineering techniques. Suitable 1,5-diaminopentane transporters may be derived from any available source including for example, Escherichia coli.

### Illustrative Amino Acid and Nucleotide Sequences

The following table identifies amino acid and nucleotide sequences used in Example 1. The corresponding sequences are shown in the Sequence Listing.

SEQ ID NO Cross-Reference Table

| | Specified | | | | | AA SEQ | DNA SEQ | Contained in | |
|---|---|---|---|---|---|---|---|---|---|
| UniProt_ID | CodonOpt | CodonOpt | Published Enzyme Name | GO MF name | Source-Organism | ID NO: | ID NO: | Strain(s) | Tested In |
| A0A0A1U6Y7 | CG | 22000202167 | BiodegRadative arginine | lysine decarboxylase | Entamoeba invadens IP1 | 1 | 150 | CgCADAV_118 | Cg only |
| | | | decarboxylase, putative | activity | | | | | |
| A0A0U9HDH2 | YL | 22000483434 | Lysine decarboxylase | catalytic activity | Tepidanaerobacter syntrophicus | 2 | 151 | | |
| A0A0A1VRH6 | CG | 22000202167 | Lysine decarboxylase | lysine decarboxylase | Microcystis aeruginosa NIES-44 | 3 | 152 | | |
| | | | | activity | | | | | |
| Q81MS2 | CG | 22000202167 | Arginine decarboxylase | arginine decarboxylase | Bacillus anthracis | 4 | 153 | | |
| | | | | activity; lysine | | | | | |
| | | | | decarboxylase activity | | | | | |
| A0A1J4RBD5 | BS | 22000483450 | Lysine decarboxylase | catalytic activity | Salmonella enterica I | 5 | 154 | | |
| F4MZD9 | YL | 22000483434 | Lysine decarboxylase, | lysine decarboxylase | Yersinia enterocolitica W22703 | 6 | 155 | | |
| | | | constitutive | activity; ornithine | | | | | |
| | | | | decarboxylase activity | | | | | |
| D8GWH5 | CG | 22000202167 | Lysine decarboxylase | lysine decarboxylase | Bacillus cereus var. anthracis | 7 | 156 | CgCADAV_97 | Cg only |
| | | | | activity | (strain CI) | | | | |
| A0A1M7RI96 | YL | 22000483434 | Lysine decarboxylase | catalytic activity | Cryptosporangium aurantiacum | 8 | 157 | | |
| A0A1T4P6M4 | BS | 22000483450 | Lysine decarboxylase | catalytic activity | Garciella nitratireducens DSM | 9 | 158 | BsCADAV_01 | Bs only |
| | | | | | 15102 | | | | |
| G8SKC2 | CG | 22000202167 | Lysine decarboxylase | lysine decarboxylase | Actinoplanes sp. (strain ATCC | 10 | 159 | CgCADAV_98 | Cg only |
| | | | | activity | 31044/CBS 674.73/SE50/110) | | | | |
| P0A9H4 | BS | 22000483450 | Inducible lysine | lysine decarboxylase | Escherichia coli O157:H7 | 11 | 160 | | |
| | | | decarboxylase | activity | | | | | |
| B1XVH2 | BS | 22000483450 | Lysine decarboxylase | lysine decarboxylase | Polynucleobacter necessarius | 12 | 161 | BsCADAV_04 | Bs only |
| | | | | activity | subsp. necessarius (strain STIR1) | | | | |
| A0A1G9YTS7 | CG | 22000202167 | Lysine decarboxylase | catalytic activity | Sediminibacillus halophilus | 13 | 162 | | |
| A0A1T4QL79 | BS | 22000483450 | Lysine decarboxylase | catalytic activity | Carboxydocella sporoproducens | 14 | 163 | | |
| | | | | | DSM 16521 | | | | |
| R7FNV2 | YL | 22000483434 | Lysine decarboxylase | catalytic activity | Clostridium sp. CAG:288 | 15 | 164 | | |
| A0A0H3KNM1 | CG | 22000202167 | Lysine decarboxylase | lysine decarboxylase | Burkholderia multivorans (strain | 16 | 165 | | |
| | | | | activity | ATCC 17616/249) | | | | |
| E0NW26 | CG | 22000202167 | Orn/Lys/Arg | lysine decarboxylase | Selenomonas sp. oral taxon 149 | 17 | 166 | CgCADAV_132 | Cg only |
| | | | decarboxylase, major | activity | str. 67H29BP | | | | |
| | | | domain protein | | | | | | |
| F4MZD9 | BS | 22000483450 | Lysine decarboxylase, | lysine decarboxylase | Yersinia enterocolitica W22703 | 6 | 167 | BsCADAV_02 | Bs only |
| | | | constitutive | activity; ornithine | | | | | |
| | | | | decarboxylase activity | | | | | |
| A0A0H3B5Z1 | CG | 22000202167 | Lysine decarboxylase | lysine decarboxylase | Yersinia pseudotuberculosis | 18 | 168 | CgCADAV_99 | Cg only |
| | | | | activity | serotype O:3 (strain YPIII) | | | | |
| U5SA13 | YL | 22000483434 | Lysine decarboxylase | catalytic activity | Carnobacterium inhibens subsp. | 19 | 169 | | |
| | | | | | gilichinskyi | | | | |
| A0A1C6W736 | CG | 22000202167 | Lysine decarboxylase | lysine decarboxylase | Bacillus cytotoxicus | 20 | 170 | CgCADAV_120 | Cg only |
| | | | | activity | | | | | |
| W0HLJ4 | YL | 22000483434 | Lysine decarboxylase | lysine decarboxylase | Candidatus Sodalis pierantonius | 21 | 171 | | |
| | | | inducible | activity | str. SOPE | | | | |
| R6FYX1 | BS | 22000483450 | Arginine/lysine | catalytic activity | Clostridium sp. CAG:221 | 22 | 172 | BsCADAV_05 | Bs only |
| | | | decarboxylase | | | | | | |
| A0A1B4WVT4 | CG | 22000202167 | Lysine decarboxylase | lysine decarboxylase | Pseudomonas sp. LAB-08 | 23 | 173 | CgCADAV_100 | Cg only |
| | | | | activity | | | | | |
| W8X0P9 | YL | 22000483434 | Arginine decarboxylase | arginine decarboxylase | Castellaniella defragrans 65Phen | 24 | 174 | | |
| | | | Ornithine decarboxylase | activity; lysine | | | | | |
| | | | Lysine decarboxylase | decarboxylase activity; | | | | | |
| | | | | ornithine decarboxylase | | | | | |
| | | | | activity | | | | | |
| A0A1T4P6M4 | CG | 22000202167 | Lysine decarboxylase | catalytic activity | Garciella nitratireducens DSM | 9 | 175 | CgCADAV_77 | Cg only |
| | | | | | 15102 | | | | |
| A0A0A3ITC5 | CG | 22000202167 | Lysine decarboxylase | catalytic activity | Lysinibacillus odysseyi 34hs-1 = | 25 | 176 | CgCADAV_82 | Cg only |
| | | | | | NBRC 100172 | | | | |
| G8AE67 | CG | 22000202167 | Lysine/ornithine | lysine decarboxylase | Azospirillum brasilense Sp245 | 26 | 177 | CgCADAV_83 | Cg only |
| | | | decarboxylase | activity; ornithine | | | | | |
| | | | | decarboxylase activity | | | | | |
| D5AMW9 | CG | 22000202167 | Lysine/ornithine | lysine decarboxylase | Rhodobacter capsulatus (strain | 27 | 178 | CgCADAV_119 | Cg only |
| | | | decarboxylase | activity; ornithine | ATCC BAA-309/NBRC 16581/SB1003) | | | | |
| | | | | decarboxylase activity | | | | | |
| G7EXN9 | CG | 22000202167 | Lysine decarboxylase | lysine decarboxylase | Pseudoalteromonas sp. BSi20311 | 28 | 179 | CgCADAV_121 | Cg only |
| | | | | activity | | | | | |
| R6FYX1 | CG | 22000202167 | Arginine/lysine | catalytic activity | Clostridium sp. CAG:221 | 22 | 180 | CgCADAV_79 | Cg only |
| | | | decarboxylase | | | | | | |
| A0A245ZEH3 | CG | 22000202167 | Lysine/ornithine | lysine decarboxylase | Sphingomonas mucosissima | 29 | 181 | CgCADAV_100 | Cg only |
| | | | decarboxylase | activity; ornithine | | | | | |
| | | | | decarboxylase activity | | | | | |
| D7GUC4 | CG | 22000202167 | Arginine decarboxylase | arginine decarboxylase | butyrate-producing bacterium SS3/4 | 30 | 182 | CgCADAV_107 | Cg only |
| | | | | activity; lysine | | | | | |
| | | | | decarboxylase activity | | | | | |
| A0A1J0KV28 | CG | 22000202167 | Lysine decarboxylase, | lysine decarboxylase | Francisella sp. CA97-1460 | 31 | 183 | CgCADAV_97 | Cg only |
| | | | inducible | activity | | | | | |
| M8CMT6 | CG | 22000202167 | Arginine/lysine/ornithine | lysine decarboxylase | Thermoanaerobacter | 32 | 184 | CgCADAV_101 | Cg only |
| | | | decarboxylase | activity | thermohydrosulfuricus WC1 | | | | |
| A0A1D7W8T4 | YL | 22000483434 | Arginine decarboxylase | arginine decarboxylase | Brevibacterium linens | 33 | 185 | | |
| | | | | activity; lysine | | | | | |
| | | | | decarboxylase activity | | | | | |
| A0A011NX48 | YL | 22000483434 | Lysine decarboxylase LdcC | catalytic activity | Candidatus Accumulibacter sp. | 34 | 186 | | |
| | | | | | BA-94 | | | | |
| N4WSH8 | CG | 22000202167 | Lysine decarboxylase | catalytic activity | Gracilibacillus halophilus | 35 | 187 | CgCADAV_87 and | Cg & Sc |
| | | | | | YIM-C55.5 | | | ScCADAV_80 | |
| A0A240CR45 | CG | 22000202167 | Lysine decarboxylase LdcC | lysine decarboxylase | Eikenella corrodens | 36 | 188 | | |
| | | | | activity | | | | | |
| B6INL8 | CG | 22000202167 | Lysine | lysine decarboxylase | Rhodospirillum centenum (strain | 37 | 189 | | |
| | | | | activity; ornithine | ATCC 51521/SW) | | | | |
| | | | | decarboxylase activity | | | | | |
| A0A1M6PLW1 | CG | 22000202167 | Lysine decarboxylase | catalytic activity | Anaerobranca californiensis DSM | 38 | 190 | | |
| | | | | | 14826 | | | | |
| A0A150JTY7 | CG | 22000202167 | Arginine decarboxylase | arginine decarboxylase | Bacillus coagulans | 39 | 191 | CgCADAV_102 | Cg only |
| | | | | activity; lysine | | | | | |
| | | | | decarboxylase activity | | | | | |
| Q7NFN7 | BS | 22000483450 | Lysine decarboxylase | catalytic activity | Gloeobacter violaceus (strain | 40 | 192 | BsCADAV_03 | Bs only |
| | | | | | PCC 7421) | | | | |
| A0A1A8VMS3 | CG | 22000202167 | Lysine decarboxylase, | catalytic activity | Plasmodium malariae | 41 | 193 | | |
| | | | putative | | | | | | |
| A0A0A2BEA5 | CG | 22000202167 | Lysine decarboxylase | lysine decarboxylase | Prochlorococcus sp. MIT 0601 | 42 | 194 | CgCADAV_101 | Cg only |
| | | | | activity | | | | | |
| A0A011NX48 | CG | 22000202167 | Lysine decarboxylase LdcC | catalytic activity | Candidatus Accumulibacter sp. | 34 | 195 | CgCADAV_78 | Cg only |
| | | | | | BA-94 | | | | |
| D5D940 | CG | 22000202167 | Lysine decarboxylase | lysine decarboxylase | Bacillus megaterium (strain | 43 | 196 | CgCADAV_102 | Cg only |
| | | | | activity | DSM 319) | | | | |
| P52095 | YL | 22000483434 | Constitutive lysine | identical protein | Escherichia coli (strain K12) | 44 | 197 | | |
| | | | decarboxylase | binding; lysine | | | | | |
| | | | | decarboxylase activity | | | | | |
| D7DQC2 | CG | 22000202167 | Lysine decarboxylase | lysine decarboxylase | Methylotenera versatilis (strain | 45 | 198 | CgCADAV_115 | Cg only |
| | | | | activity | 301) | | | | |
| A0A060RT32 | CG | 22000202167 | Lysine decarboxylase, | lysine decarboxylase | Plasmodium reichenowi | 46 | 199 | | |
| | | | putative | activity | | | | | |
| E7SAJ9 | CG | 22000202167 | Orn/Lys/Arg | lysine decarboxylase | Streptococcus australis ATCC | 47 | 200 | CgCADAV_131 | Cg only |
| | | | decarboxylase, major | activity | 700641 | | | | |
| | | | domain protein | | | | | | |
| A0A081FVR4 | CG | 22000202167 | Arginine decarboxylase | arginine decarboxylase | Marinobacterium sp. AK27 | 48 | 201 | CgCADAV_83 | Cg only |
| | | | | activity; lysine | | | | | |
| | | | | decarboxylase activity; | | | | | |
| | | | | ornithine decarboxylase | | | | | |
| | | | | activity | | | | | |
| R7B1X0 | CG | 22000202167 | Lysine decarboxylase | catalytic activity | Bacteroides pectinophilus CAG:437 | 49 | 202 | CgCADAV_93 and | Cg & Sc |
| | | | | | | | | ScCADAV_85 | |
| B3PWQ0 | BS | 22000483450 | Probable lysine | lysine decarboxylase | Rhizobium etli (strain CIAT 652) | 50 | 203 | | |
| | | | decarboxylase protein | activity | | | | | |
| B9YZ77 | CG | 22000202167 | Lysine decarboxylase | lysine decarboxylase | Pseudogulbenkiania ferrooxidans | 51 | 204 | | |
| | | | | activity | 2002 | | | | |
| D4KTI3 | CG | 22000202167 | Arginine/lysine/ornithine | lysine decarboxylase | Roseburia intestinalis M50/1 | 52 | 205 | CgCADAV_105 | Cg only |
| | | | decarboxylases | activity | | | | | |
| D4KVB9 | CG | 22000202167 | Arginine decarboxylase | arginine decarboxylase | Roseburia intestinalis XB6B4 | 53 | 206 | CgCADAV_116 | Cg only |
| | | | | activity; lysine | | | | | |
| | | | | decarboxylase activity | | | | | |
| U5SA13 | BS | 22000483450 | Lysine decarboxylase | catalytic activity | Carnobacterium inhibens subsp. | 19 | 207 | | |
| | | | | | gilichinskyi | | | | |
| A0A1A8VN60 | CG | 22000202167 | Lysine decarboxylase, | catalytic activity | Plasmodium ovale curtisi | 54 | 208 | | |
| | | | putative | | | | | | |
| R6Y4K0 | YL | 22000483434 | Lysine decarboxylase | catalytic activity | Firmicutes bacterium CAG:345 | 55 | 209 | | |
| R6Y4K0 | BS | 22000483450 | Lysine decarboxylase | catalytic activity | Firmicutes bacterium CAG:345 | 55 | 210 | | |
| B5INA8 | CG | 22000202167 | Orn/Lys/Arg | lysine decarboxylase | Cyanobium sp. PCC 7001 | 56 | 211 | CgCADAV_100 | Cg only |
| | | | decarboxylases family 1 | activity | | | | | |
| A0A090NAB7 | CG | 22000202167 | Lysine decarboxylase | lysine decarboxylase | Shigella dysenteriae WRSd3 | 57 | 212 | CgCADAV_112 | Cg only |
| | | | | activity | | | | | |
| R7HED2 | BS | 22000483450 | Lysine decarboxylase | catalytic activity | Eubacterium sp. CAG:38 | 58 | 213 | BsCADAV_01 | Bs only |
| A0A0C4YL17 | CG | 22000202167 | Lysine decarboxylase | lysine decarboxylase | Cupriavidus basilensis | 59 | 214 | CgCADAV_113 | Cg only |
| | | | | activity | | | | | |
| R7FNV2 | CG | 22000202167 | Lysine decarboxylase | catalytic activity | Clostridium sp. CAG:288 | 15 | 215 | CgCADAV_79 | Cg only |
| A0A1T4QL79 | CG | 22000202167 | Lysine decarboxylase | catalytic activity | Carboxydocella sporoproducens DSM | 14 | 216 | | |
| | | | | | 16521 | | | | |
| G8AE67 | YL | 22000483434 | Lysine/ornithine | lysine decarboxylase | Azospirillum brasilense Sp245 | 26 | 217 | | |
| | | | decarboxylase | activity; ornithine | | | | | |
| | | | | decarboxylase activity | | | | | |
| A0A1J4RBD5 | CG | 22000202167 | Lysine decarboxylase | catalytic activity | Salmonella enterica I | 5 | 218 | CgCADAV_78 | Cg only |
| K2FIN6 | BS | 22000483450 | Lysine decarboxylase | catalytic activity | Salimicrobium jeotgali | 60 | 219 | | |
| A8GLC5 | CG | 22000202167 | Lysine decarboxylase | lysine decarboxylase | Serratia proteamaculans (strain | 61 | 220 | CgCADAV_99 | Cg only |
| | | | | activity | 568) | | | | |
| A0A1V0SRU9 | YL | 22000483434 | Lysine decarboxylase | catalytic activity | Sporosarcina ureae | 62 | 221 | | |
| A0A077YCW2 | CG | 22000202167 | Lysine decarboxylase, | lysine decarboxylase | Plasmodium berghei (strain Anka) | 63 | 222 | | |
| | | | putative | activity | | | | | |
| A0A1N6T1T8 | CG | 22000202167 | L-lysine decarboxylase | carboxy-lyase | Aeromonas veronii | 64 | 223 | CgCADAV_80 | Cg only |
| | | | | activity | | | | | |
| B9YZ77 | BS | 22000483450 | Lysine decarboxylase | lysine decarboxylase | Pseudogulbenkiania ferrooxidans | 51 | 224 | | |
| | | | | activity | 2002 | | | | |
| D8NR11 | CG | 22000202167 | Lysine decarboxylase | lysine decarboxylase | Ralstonia solanacearum CFBP2957 | 65 | 225 | CgCADAV_132 | Cg only |
| | | | | activity | | | | | |
| E8UEY5 | BS | 22000483450 | Arginine, Ornithine and | arginine decarboxylase | Taylorella equigenitalis (strain | 66 | 226 | | |
| | | | Lysine decarboxylase | activity; lysine | MCE9) | | | | |
| | | | | decarboxylase activity; | | | | | |
| | | | | ornithine decarboxylase | | | | | |
| | | | | activity | | | | | |
| A0A1M7RI96 | CG | 22000202167 | Lysine decarboxylase | catalytic activity | Cryptosporangium aurantiacum | 8 | 227 | CgCADAV_85 | Cg only |
| W0HLJ4 | BS | 22000483450 | Lysine decarboxylase | lysine decarboxylase | Candidatus Sodalis pierantonius | 21 | 228 | BsCADAV_04 | Bs only |
| | | | inducible | activity | str. SOPE | | | | |
| W0HLJ4 | CG | 22000202167 | Lysine decarboxylase | lysine decarboxylase | Candidatus Sodalis pierantonius | 21 | 229 | ScCADAV_76 | Sc only |
| | | | inducible | activity | str. SOPE | | | | |
| A0A101I516 | CG | 22000202167 | Arginine decarboxylase | catalytic activity | candidate division TA06 bacterium | 67 | 230 | CgCADAV_82 | Cg only |
| | | | Lysine decarboxylase | | 34_109 | | | | |
| | | | Ornithine decarboxylase | | | | | | |
| Q8I1X1 | CG | 22000202167 | Lysine decarboxylase, | lysine decarboxylase | Plasmodium falciparum (isolate | 68 | 231 | | |
| | | | putative | activity | 3D7) | | | | |
| B6JAY1 | CG | 22000202167 | Lysine/ornithine | lysine decarboxylase | Oligotropha carboxidovorans | 69 | 232 | CgCADAV_122 | Cg only |
| | | | decarboxylase Ldc | activity; ornithine | (strain ATCC 49405/DSM 1227/ | | | | |
| | | | | decarboxylase activity | KCTC 32145/OM5) | | | | |
| Q2JVN4 | CG | 22000202167 | Orn/Lys/Arg decarboxylase | catalytic activity | Synechococcus sp. (strain | 70 | 233 | CgCADAV_93 and | Cg & Sc |
| | | | | | JA-3-3Ab) (Cyanobacteria | | | ScCADAV_85 | |
| | | | | | bacterium Yellowstone A-Prime) | | | | |
| K4ZQR8 | CG | 22000202167 | Lysine decarboxylase | lysine decarboxylase | Paenibacillus alvei DSM 29 | 71 | 234 | CgCADAV_83 | Cg only |
| | | | YaaO | activity | | | | | |
| A0A1A9AX65 | CG | 22000202167 | Lysine decarboxylase, | lysine decarboxylase | Plesiomonas shigelloides | 72 | 235 | | |
| | | | inducible | activity | (Aeromonas shigelloides) | | | | |
| A0A011NX48 | BS | 22000483450 | Lysine decarboxylase LdcC | catalytic activity | Candidatus Accumulibacter sp. | 34 | 236 | | |
| | | | | | BA-94 | | | | |
| Q2JVN4 | BS | 22000483450 | Orn/Lys/Arg decarboxylase | catalytic activity | Synechococcus sp. (strain | 70 | 237 | BsCADAV_03 | Bs only |
| | | | | | JA-3-3Ab) (Cyanobacteria | | | | |
| | | | | | bacterium Yellowstone A-Prime) | | | | |
| A0A1M4T9I0 | BS | 22000483450 | Lysine decarboxylase | catalytic activity | Alkalibacter saccharofermentans | 73 | 238 | | |
| | | | | | DSM 14828 | | | | |
| B4SMN4 | CG | 22000202167 | Lysine decarboxylase | lysine decarboxylase | Stenotrophomonas maltophilia | 74 | 239 | CgCADAV_106 | Cg only |
| | | | | activity | (strain R551-3) | | | | |
| A0A1M6PK15 | YL | 22000483434 | Lysine decarboxylase | catalytic activity | Alicyclobacillus sp. USBA-503 | 75 | 240 | | |
| A0A1G4GTM1 | CG | 22000202167 | Lysine decarboxylase-like | lysine decarboxylase | Plasmodium vivax | 76 | 241 | CgCADAV_122 | Cg only |
| | | | protein, putative | activity | | | | | |
| N4WSH8 | YL | 22000483434 | Lysine decarboxylase | catalytic activity | Gracilibacillus halophilus | 35 | 242 | | |
| | | | | | YIM-C55.5 | | | | |
| K4ZQR8 | YL | 22000483434 | Lysine decarboxylase | lysine decarboxylase | Paenibacillus alvei DSM 29 | 71 | 243 | | |
| | | | YaaO | activity | | | | | |
| L8AGJ7 | CG | 22000202167 | Lysine decarboxylase | catalytic activity | Bacillus subtilis BEST7613 | 77 | 244 | CgCADAV_85 | Cg only |
| A0A1Y0Y9X9 | CG | 22000202167 | Lysine decarboxylase | lysine decarboxylase | Bacillus licheniformis | 78 | 245 | CgCADAV_111 | Cg only |
| | | | | activity | | | | | |
| A0A150LIS5 | YL | 22000483434 | Arginine decarboxylase | arginine decarboxylase | Anoxybacillus flavithermus | 79 | 246 | | |
| | | | | activity; lysine | | | | | |
| | | | | decarboxylase activity | | | | | |
| A0A1M4RP40 | CG | 22000202167 | Arginine decarboxylase/ | arginine decarboxylase | Staphylococcus aureus | 80 | 247 | CgCADAV_79 | Cg only |
| | | | Lysine decarboxylase | activity | | | | | |
| R7FNV2 | BS | 22000483450 | Lysine decarboxylase | catalytic activity | Clostridium sp. CAG:288 | 15 | 248 | BsCADAV_05 | Bs only |
| R6Y4K0 | CG | 22000202167 | Lysine decarboxylase | catalytic activity | Firmicutes bacterium CAG:345 | 55 | 249 | | |
| A0A1D7VZF2 | CG | 22000202167 | Arginine decarboxylase | arginine decarboxylase | Brevibacterium linens | 81 | 250 | CgCADAV_105 | Cg only |
| | | | | activity; lysine | | | | | |
| | | | | decarboxylase activity | | | | | |
| A8I481 | BS | 22000483450 | Lysine decarboxylase | catalytic activity | Chlamydomonas reinhardtii | 82 | 251 | | |
| | | | | | (Chlamydomonas smithii) | | | | |
| A0A1T4QL79 | YL | 22000483434 | Lysine decarboxylase | catalytic activity | Carboxydocella sporoproducens | 14 | 252 | | |
| | | | | | DSM 16521 | | | | |
| A0A150MS57 | CG | 22000202167 | Arginine decarboxylase | arginine decarboxylase | Geobacillus sp. B4113_201601 | 83 | 253 | CgCADAV_121 | Cg only |
| | | | | activity; lysine | | | | | |
| | | | | decarboxylase activity | | | | | |
| R7HED2 | CG | 22000202167 | Lysine decarboxylase | catalytic activity | Eubacterium sp. CAG:38 | 58 | 254 | CgCADAV_77 | Cg only |
| A0A1G9YTS7 | BS | 22000483450 | Lysine decarboxylase | catalytic activity | Sediminibacillus halophilus | 13 | 255 | | |
| A0A0A3ITC5 | BS | 22000483450 | Lysine decarboxylase | catalytic activity | Lysinibacillus odysseyi | 25 | 256 | | |
| | | | | | 34hs-1 = NBRC 100172 | | | | |
| L8AGJ7 | BS | 22000483450 | Lysine decarboxylase | catalytic activity | Bacillus subtilis BEST7613 | 77 | 257 | | |
| Q7NFN7 | YL | 22000483434 | Lysine decarboxylase | catalytic activity | Gloeobacter violaceus (strain | 40 | 258 | | |
| | | | | | PCC 7421) | | | | |
| E1RF59 | CG | 22000202167 | Lysine decarboxylase | lysine decarboxylase | Methanolacinia petrolearia | 84 | 259 | CgCADAV_106 | Cg only |
| | | | | activity | (strain DSM 11571/OCM 486/ | | | | |
| | | | | | SEBR 4847) ((Methanoplanus | | | | |
| | | | | | petrolearius) | | | | |
| U6L990 | CG | 22000202167 | Lysine decarboxylase, | catalytic activity | Eimeria brunetti | 85 | 260 | CgCADAV_87 and | Cg & Sc |
| | | | putative | | | | | ScCADAV_80 | |
| F4MZD9 | CG | 22000202167 | Lysine decarboxylase, | lysine decarboxylase | Yersinia enterocolitica W22703 | 6 | 261 | CgCADAV_88 and | Cg & Sc |
| | | | constitutive | activity; ornithine | | | | ScCADAV_81 | |
| | | | | decarboxylase activity | | | | | |
| B1XVH2 | YL | 22000483434 | Lysine decarboxylase | lysine decarboxylase | Polynucleobacter necessarius | 12 | 262 | | |
| | | | | activity | subsp. necessarius (strain | | | | |
| | | | | | STIR1) | | | | |
| A0A1C3KA53 | CG | 22000202167 | Lysine decarboxylase, | lysine decarboxylase | Plasmodium malariae | 86 | 263 | | |
| | | | putative | activity | | | | | |
| E9TK07 | CG | 22000202167 | Lysine decarboxylase, | lysine decarboxylase | Escherichia coli MS 117-3 | 87 | 264 | CgCADAV_107 | Cg only |
| | | | inducible | activity | | | | | |
| A0A081FVR4 | BS | 22000483450 | Arginine decarboxylase | arginine decarboxylase | Marinobacterium sp. AK27 | 48 | 265 | | |
| | | | | activity; lysine | | | | | |
| | | | | decarboxylase activity; | | | | | |
| | | | | ornithine decarboxylase | | | | | |
| | | | | activity | | | | | |
| A0A212LWY4 | CG | 22000202167 | Lysine/ornithine | lysine decarboxylase | uncultured Sporomusa sp. | 88 | 266 | CgCADAV_98 | Cg only |
| | | | decarboxylase | activity; ornithine | | | | | |
| | | | | decarboxylase activity | | | | | |
| A0A1A8VN60 | YL | 22000483434 | Lysine decarboxylase, | catalytic activity | Plasmodium ovale curtisi | 54 | 267 | | |
| | | | putative | | | | | | |
| A0A1M6CES8 | BS | 22000483450 | Lysine decarboxylase | catalytic activity | Dethiosulfatibacter aminovorans | 89 | 268 | | |
| | | | | | DSM 17477 | | | | |
| A0A0A2ARD9 | BS | 22000483450 | Lysine decarboxylase | lysine decarboxylase | Prochlorococcus marinus str. | 90 | 269 | BsCADAV_02 | Bs only |
| | | | | activity | MIT 9314 | | | | |
| A0A1M7RI96 | BS | 22000483450 | Lysine decarboxylase | catalytic activity | Cryptosporangium aurantiacum | 8 | 270 | | |
| B3KZY7 | CG | 22000202167 | Lysine decarboxylase, | lysine decarboxylase | Plasmodium knowlesi (strain H) | 91 | 271 | CgCADAV_108 | Cg only |
| | | | putative | activity | | | | | |
| V5AFU2 | YL | 22000483434 | Lysine decarboxylase, | lysine decarboxylase | Betaproteobacteria bacterium | 92 | 272 | | |
| | | | inducible | activity | MOLA814 | | | | |
| K2FIN6 | CG | 22000202167 | Lysine decarboxylase | catalytic activity | Salimicrobium jeotgali | 60 | 273 | | |
| A0A1N6T1T8 | BS | 22000483450 | L-lysine decarboxylase | carboxy-lyase activity | Aeromonas veronii | 64 | 274 | | |
| A0A0U9HDH2 | BS | 22000483450 | Lysine decarboxylase | catalytic activity | Tepidanaerobacter syntrophicus | 2 | 275 | | |
| A0A1J5S026 | CG | 22000202167 | Lysine/ornithine | lysine decarboxylase | mine drainage metagenome | 93 | 276 | | |
| | | | decarboxylase | activity; ornithine | | | | | |
| | | | | decarboxylase activity | | | | | |
| A0A1A9AX65 | YL | 22000483434 | Lysine decarboxylase, | lysine decarboxylase | Plesiomonas shigelloides | 72 | 277 | | |
| | | | inducible | activity | (Aeromonas shigelloides) | | | | |
| G8AE67 | BS | 22000483450 | Lysine/ornithine | lysine decarboxylase | Azospirillum brasilense Sp245 | 26 | 278 | | |
| | | | decarboxylase | activity; ornithine | | | | | |
| | | | | decarboxylase activity | | | | | |
| A0A031HSL8 | CG | 22000202167 | Lysine decarboxylase | lysine decarboxylase | Delftia sp. RIT313 | 94 | 279 | CgCADAV_116 and | Cg only |
| | | | | activity | | | | CgCADAV_131 | |
| A0A1M6PK15 | BS | 22000483450 | Lysine decarboxylase | catalytic activity | Alicyclobacillus sp. USBA-503 | 75 | 280 | BsCADAV_01 | Bs only |
| B0KMZ8 | CG | 22000202167 | Lysine decarboxylase | lysine decarboxylase | Pseudomonas putida (strain GB-1) | 95 | 281 | CgCADAV_97 | Cg only |
| | | | | activity | | | | | |
| A0A081FVR4 | YL | 22000483434 | Arginine decarboxylase | arginine decarboxylase | Marinobacterium sp. AK27 | 48 | 282 | | |
| | | | | activity; lysine | | | | | |
| | | | | decarboxylase activity; | | | | | |
| | | | | ornithine decarboxylase | | | | | |
| | | | | activity | | | | | |
| A0A191W896 | BS | 22000483450 | Lysine decarboxylase | catalytic activity | Vibrio anguillarum (Listonella | 96 | 283 | | |
| | | | | | anguillarum) | | | | |
| A0A191W896 | YL | 22000483434 | Lysine decarboxylase | catalytic activity | Vibrio anguillarum (Listonella | 96 | 284 | | |
| | | | | | anguillarum) | | | | |
| A0A1M6CES8 | CG | 22000202167 | Lysine decarboxylase | catalytic activity | Dethiosulfatibacter aminovorans | 89 | 285 | CgCADAV_80 | Cg only |
| | | | | | DSM 17477 | | | | |
| A0A0A2ARD9 | YL | 22000483434 | Lysine decarboxylase | lysine decarboxylase | Prochlorococcus marinus str. | 90 | 286 | | |
| | | | | activity | MIT 9314 | | | | |
| A0A0L0TNR8 | CG | 22000202167 | Arginine decarboxylase | arginine decarboxylase | Candidatus Burkholderia crenata | 97 | 287 | CgCADAV_107 | Cg only |
| | | | Ornithine decarboxylase | activity; lysine | | | | | |
| | | | Lysine decarboxylase | decarboxylase activity; | | | | | |
| | | | | ornithine decarboxylase | | | | | |
| | | | | activity | | | | | |
| A0A1R4HN10 | CG | 22000202167 | Arginine decarboxylase/ | arginine decarboxylase | Leucobacter sp. 7(1) | 98 | 288 | | |
| | | | Lysine decarboxylase | activity; lysine | | | | | |
| | | | | decarboxylase activity | | | | | |
| A0A0M2YBA0 | YL | 22000483434 | Lysine decarboxylase LdcC | lysine decarboxylase | Pantoea ananas (Erwinia uredovora) | 99 | 289 | | |
| | | | | activity | | | | | |
| A0A168T111 | CG | 22000202167 | Lysine decarboxylase | catalytic activity | Phormidium willei BDU 130791 | 100 | 290 | CgCADAV_89 | Cg only |
| X5JQV6 | CG | 22000202167 | Lysine decarboxylase | lysine decarboxylase | Richelia intracellularis | 101 | 291 | CgCADAV_111 | Cg only |
| | | | | activity | | | | | |
| A0A077LYA4 | CG | 22000202167 | Lysine decarboxylase | lysine decarboxylase | Tetrasphaera japonica T1-X7 | 102 | 292 | CgCADAV_112 | Cg only |
| | | | | activity | | | | | |
| A0A0A5GAB3 | YL | 22000483434 | Lysine decarboxylase | catalytic activity | Pontibacillus halophilus JSM | 103 | 293 | | |
| | | | | | 076056 = DSM 19796 | | | | |
| A0A089QSV4 | CG | 22000202167 | Lysine decarboxylase | lysine decarboxylase | Prochlorococcus sp. MIT 0801 | 104 | 294 | | |
| | | | | activity | | | | | |
| U6L990 | BS | 22000483450 | Lysine decarboxylase, | catalytic activity | Eimeria brunetti | 85 | 295 | | |
| | | | putative | | | | | | |
| F7S7C7 | CG | 22000202167 | Orn/DAP/Arg | lysine decarboxylase | Acidiphilium sp. PM | 105 | 296 | CgCADAV_112 | Cg only |
| | | | decarboxylase 2 | activity; ornithine | | | | | |
| | | | | decarboxylase activity | | | | | |
| B3PWQ0 | YL | 22000483434 | Probable lysine | lysine decarboxylase | Rhizobium etli (strain CIAT 652) | 50 | 297 | | |
| | | | decarboxylase protein | activity | | | | | |
| N1JS60 | CG | 22000202167 | Lysine decarboxylase | lysine decarboxylase | Mesotoga infera | 106 | 298 | CgCADAV_118 | Cg only |
| | | | | activity | | | | | |
| E8LF60 | CG | 22000202167 | Lysine/ornithine | lysine decarboxylase | Phascolarctobacterium | 107 | 299 | CgCADAV_113 | Cg only |
| | | | decarboxylase | activity; ornithine | succinatutens YIT 12067 | | | | |
| | | | | decarboxylase activity | | | | | |
| A0A086CIE7 | CG | 22000202167 | Arginine decarboxylase | lysine decarboxylase | Candidatus Atelocyanobacterium | 108 | 300 | CgCADAV_98 | Cg only |
| | | | | activity | thalassa isolate SIO64986 | | | | |
| D5X169 | CG | 22000202167 | Lysine decarboxylase | lysine decarboxylase | Thiomonas intermedia (strain K12) | 109 | 301 | CgCADAV_120 | Cg only |
| | | | | activity | (Thiobacillus intermedius) | | | | |
| B9YZ77 | YL | 22000483434 | Lysine decarboxylase | lysine decarboxylase | Pseudogulbenkiania ferrooxidans | 51 | 302 | | |
| | | | | activity | 2002 | | | | |
| Q7U7N7 | CG | 22000202167 | Orn/Lys/Arg | lysine decarboxylase | Synechococcus sp. (strain WH8102) | 110 | 303 | CgCADAV_111 | Cg only |
| | | | decarboxylases family 1 | activity | | | | | |
| A0A0N1FV26 | CG | 22000202167 | Lysine decarboxylase | lysine decarboxylase | Actinobacteria bacterium OV450 | 111 | 304 | CgCADAV_129 | Cg only |
| | | | | activity | | | | | |
| A0A1V0SRU9 | BS | 22000483450 | Lysine decarboxylase | catalytic activity | Sporosarcina ureae | 62 | 305 | | |
| Q7V108 | CG | 22000202167 | Orn/Lys/Arg | lysine decarboxylase | Prochlorococcus marinus subsp. | 112 | 306 | | |
| | | | decarboxylases family 1 | activity | pastoris (strain CCMP1986/NIES- | | | | |
| | | | | | 2087/MED4) | | | | |
| A0A089PLU5 | CG | 22000202167 | Lysine decarboxylase | lysine decarboxylase | Pluralibacter gergoviae | 113 | 307 | CgCADAV_115 | Cg only |
| | | | | activity | (Enterobacter gergoviae) | | | | |
| A0A097EQU8 | CG | 22000202167 | Lysine decarboxylase LdcC | lysine decarboxylase | Francisella sp. FSC1006 | 114 | 308 | CgCADAV_130 | Cg only |
| | | | | activity | | | | | |
| U5SA13 | CG | 22000202167 | Lysine decarboxylase | catalytic activity | Carnobacterium inhibens subsp. | 19 | 309 | CgCADAV_89 | Cg only |
| | | | | | gilichinskyi | | | | |
| A0A1L8CVK5 | BS | 22000483450 | Lysine decarboxylase | catalytic activity | Carboxydothermus pertinax | 115 | 310 | | |
| A0A1M6PLW1 | YL | 22000483434 | Lysine decarboxylase | catalytic activity | Anaerobranca californiensis | 38 | 311 | | |
| | | | | | DSM 14826 | | | | |
| N4WSH8 | BS | 22000483450 | Lysine decarboxylase | catalytic activity | Gracilibacillus halophilus | 35 | 312 | | |
| | | | | | YIM-C55.5 | | | | |
| P52095 | BS | 22000483450 | Constitutive lysine | identical protein | Escherichia coli (strain K12) | 44 | 313 | | |
| | | | decarboxylase | binding; lysine | | | | | |
| | | | | decarboxylase activity | | | | | |
| A0A1A9AX65 | BS | 22000483450 | Lysine decarboxylase, | lysine decarboxylase | Plesiomonas shigelloides | 72 | 314 | | |
| | | | inducible | activity | (Aeromonas shigelloides) | | | | |
| A0A1K1WST1 | BS | 22000483450 | Lysine decarboxylase | catalytic activity | Thermoactinomyces sp. DSM 45891 | 116 | 315 | | |
| A0A0A3ITC5 | YL | 22000483434 | Lysine decarboxylase | catalytic activity | Lysinibacillus odysseyi | 25 | 316 | | |
| | | | | | 34hs-1 = NBRC 100172 | | | | |
| F9EMG4 | CG | 22000202167 | Lysine decarboxylase | lysine decarboxylase | Fusobacterium nucleatum subsp. | 117 | 317 | CgCADAV_129 | Cg only |
| | | | | activity; metal ion | animalis ATCC 51191 | | | | |
| | | | | binding | | | | | |
| U6L990 | YL | 22000483434 | Lysine decarboxylase, | catalytic activity | Eimeria brunetti | 85 | 318 | | |
| | | | putative | | | | | | |
| U4KJM2 | CG | 22000202167 | Lysine decarboxylase | lysine decarboxylase | Acholeplasma palmae (strain | 118 | 319 | CgCADAV_105 | Cg only |
| | | | | activity | ATCC 49389/J233) | | | | |
| A0A1M6PK15 | CG | 22000202167 | Lysine decarboxylase | catalytic activity | Alicyclobacillus sp. USBA-503 | 75 | 320 | CgCADAV_77 | Cg only |
| A0A1M4T9I0 | CG | 22000202167 | Lysine decarboxylase | catalytic activity | Alkalibacter saccharofermentans | 73 | 321 | CgCADAV_80 | Cg only |
| | | | | | DSM 14828 | | | | |
| Q5L130 | BS | 22000483450 | Lysine decarboxylase | lysine decarboxylase | Geobacillus kaustophilus (strain | 119 | 322 | BsCADAV_04 | Bs only |
| | | | | activity | HTA426) | | | | |
| F6DKP2 | CG | 22000202167 | Lysine decarboxylase | lysine decarboxylase | Desulfotomaculum ruminis (strain | 120 | 323 | CgCADAV_119 | Cg only |
| | | | | activity | ATCC 23193/DSM 2154/NCIB 8452/DL) | | | | |
| A0A150LIS5 | BS | 22000483450 | Arginine decarboxylase | arginine decarboxylase | Anoxybacillus flavithermus | 79 | 324 | | |
| | | | | activity; lysine | | | | | |
| | | | | decarboxylase activity | | | | | |
| A0A1A8VMS3 | YL | 22000483434 | Lysine decarboxylase, | catalytic activity | Plasmodium malariae | 41 | 325 | | |
| | | | putative | | | | | | |
| K4ZQR8 | BS | 22000483450 | Lysine decarboxylase | lysine decarboxylase | Paenibacillus alvei DSM 29 | 71 | 326 | | |
| | | | YaaO | activity | | | | | |
| A0A0A1A968 | CG | 22000202167 | Arginine decarboxylase | arginine decarboxylase | Escherichia coli | 121 | 327 | CgCADAV_113 | Cg only |
| | | | | activity; lysine | | | | | |
| | | | | decarboxylase activity | | | | | |
| A0A1M6CES8 | YL | 22000483434 | Lysine decarboxylase | catalytic activity | Dethiosulfatibacter aminovorans | 89 | 328 | | |
| | | | | | DSM 17477 | | | | |
| A0A1J4RBD5 | YL | 22000483434 | Lysine decarboxylase | catalytic activity | Salmonella enterica I | 5 | 329 | | |
| A0A101I516 | YL | 22000483434 | Arginine decarboxylase | catalytic activity | candidate division TA06 bacterium | 67 | 330 | | |
| | | | Lysine decarboxylase | | 34_109 | | | | |
| | | | Ornithine decarboxylase | | | | | | |
| O50657 | CG | 22000202167 | Lysine/ornithine | lysine decarboxylase | Selenomonas ruminantium | 122 | 331 | | |
| | | | decarboxylase | activity; ornithine | | | | | |
| | | | | decarboxylase activity | | | | | |
| D2T5R1 | CG | 22000202167 | Lysine decarboxylase | lysine decarboxylase | Erwinia pyrifoliae (strain | 123 | 332 | CgCADAV_116 and | Cg only |
| | | | | activity | DSM 12163/CIP 106111/Ep16/96) | | | CgCADAV_117 | |
| Q0I358 | BS | 22000483450 | L-lysine decarboxylase | lysine decarboxylase | Haemophilus somnus (strain 129Pt) | 124 | 333 | | |
| | | | | activity | (Histophilus somni) | | | | |
| A0A1D3JIM6 | CG | 22000202167 | Lysine decarboxylase, | lysine decarboxylase | Plasmodium malariae | 125 | 334 | | |
| | | | putative | activity | | | | | |
| A0A1T4P6M4 | YL | 22000483434 | Lysine decarboxylase | catalytic activity | Garciella nitratireducens DSM | 9 | 335 | | |
| | | | | | 15102 | | | | |
| V5AFU2 | CG | 22000202167 | Lysine decarboxylase, | lysine decarboxylase | Betaproteobacteria bacterium | 92 | 336 | CgCADAV_91 | Cg only |
| | | | inducible | activity | MOLA814 | | | | |
| A0A1J1H057 | CG | 22000202167 | Lysine decarboxylase, | lysine decarboxylase | Plasmodium gallinaceum | 126 | 337 | CgCADAV_118 | Cg only |
| | | | putative | activity | | | | | |
| A0A1N6T1T8 | YL | 22000483434 | L-lysine decarboxylase | carboxy-lyase activity | Aeromonas veronii | 64 | 338 | | |
| A0A0A2BQI2 | CG | 22000202167 | Lysine decarboxylase | lysine decarboxylase | Prochlorococcus sp. MIT 0602 | 127 | 339 | CgCADAV_101 | Cg only |
| | | | | activity | | | | | |
| A0A1L8CVK5 | CG | 22000202167 | Lysine decarboxylase | catalytic activity | Carboxydothermus pertinax | 115 | 340 | CgCADAV_82 | Cg only |
| P0A9H4 | YL | 22000483434 | Inducible lysine | lysine decarboxylase | Escherichia coli O157:H7 | 11 | 341 | | |
| | | | decarboxylase | activity | | | | | |
| A0A0M2YBA0 | BS | 22000483450 | Lysine decarboxylase LdcC | lysine decarboxylase | Pantoea ananas (Erwinia uredovora) | 99 | 342 | | |
| | | | | activity | | | | | |
| O50657 | CG | 22000202167 | Lysine/ornithine | lysine decarboxylase | Selenomonas ruminantium | 128 | 343 | CgCADAV_119 | Cg only |
| | | | decarboxylase | activity; ornithine | | | | | |
| | | | | decarboxylase activity | | | | | |
| B1XVH2 | CG | 22000202167 | Lysine decarboxylase | lysine decarboxylase | Polynucleobacter necessarius | 12 | 344 | ScCADAV_76 | Sc only |
| | | | | activity | subsp. necessarius (strain STIR1) | | | | |
| A0A1M4RP40 | YL | 22000483434 | Arginine decarboxylase/ | arginine decarboxylase | Staphylococcus aureus | 80 | 345 | | |
| | | | Lysine decarboxylase | activity | | | | | |
| A0A224W715 | CG | 22000202167 | Arginine decarboxylase | arginine decarboxylase | Aquitalea magnusonii | 129 | 346 | CgCADAV_106 | Cg only |
| | | | | activity; lysine | | | | | |
| | | | | decarboxylase activity; | | | | | |
| | | | | ornithine decarboxylase | | | | | |
| | | | | activity | | | | | |
| Q0I358 | YL | 22000483434 | L-lysine decarboxylase | lysine decarboxylase | Haemophilus somnus (strain 129Pt) | 124 | 347 | | |
| | | | | activity | (Histophilus somni) | | | | |
| A0A0U9HDH2 | CG | 22000202167 | Lysine decarboxylase | catalytic activity | Tepidanaerobacter syntrophicus | 2 | 348 | | |
| I0QP51 | CG | 22000202167 | Lysine decarboxylase LdcC | lysine decarboxylase | Serratia sp. M24T3 | 130 | 349 | CgCADAV_120 | Cg only |
| | | | | activity | | | | | |
| D4JWF2 | CG | 22000202167 | Arginine decarboxylase | arginine decarboxylase | [Eubacterium] siraeum 70/3 | 131 | 350 | CgCADAV_117 | Cg only |
| | | | | activity; lysine | | | | | |
| | | | | decarboxylase activity | | | | | |
| R7B1X0 | YL | 22000483434 | Lysine decarboxylase | catalytic activity | Bacteroides pectinophilus CAG:437 | 49 | 351 | | |
| A0A0M2YBA0 | CG | 22000202167 | Lysine decarboxylase LdcC | lysine decarboxylase | Pantoea ananas (Erwinia uredovora) | 99 | 352 | CgCADAV_90 and | Cg & Sc |
| | | | | activity | | | | ScCADAV_83 | |
| D3RV51 | CG | 22000202167 | Lysine decarboxylase | lysine decarboxylase | Allochromatium vinosum (strain | 132 | 353 | CgCADAV_117 | Cg only |
| | | | | activity | ATCC 17899/DSM 180/NBRC 103801/ | | | | |
| | | | | | NCIMB 10441/D) (Chromatium vinosum) | | | | |
| A0A1D7W0C4 | CG | 22000202167 | Lysine decarboxylase | lysine decarboxylase | Brevibacterium linens | 133 | 354 | CgCADAV_108 | Cg only |
| | | | | activity | | | | | |
| A0A191W896 | CG | 22000202167 | Lysine decarboxylase | catalytic activity | Vibrio anguillarum (Listonella | 96 | 355 | CgCADAV_89 | Cg only |
| | | | | | anguillarum) | | | | |
| W8X0P9 | BS | 22000483450 | Arginine decarboxylase | arginine decarboxylase | Castellaniella defragrans 65Phen | 24 | 356 | BsCADAV_02 | Bs only |
| | | | Ornithine decarboxylase | activity; lysine | | | | | |
| | | | Lysine decarboxylase | decarboxylase activity; | | | | | |
| | | | | ornithine decarboxylase | | | | | |
| | | | | activity | | | | | |
| A0A1D7W8T4 | CG | 22000202167 | Arginine decarboxylase | arginine decarboxylase | Brevibacterium linens | 33 | 357 | CgCADAV_91 | Cg only |
| | | | | activity; lysine | | | | | |
| | | | | decarboxylase activity | | | | | |
| L8AGJ7 | YL | 22000483434 | Lysine decarboxylase | catalytic activity | Bacillus subtilis BEST7613 | 77 | 358 | | |
| R7B1X0 | BS | 22000483450 | Lysine decarboxylase | catalytic activity | Bacteroides pectinophilus CAG:437 | 49 | 359 | BsCADAV_03 | Bs only |
| A0A1M6PLW1 | BS | 22000483450 | Lysine decarboxylase | catalytic activity | Anaerobranca californiensis DSM | 38 | 360 | | |
| | | | | | 14826 | | | | |
| K2FIN6 | YL | 22000483434 | Lysine decarboxylase | catalytic activity | Salimicrobium jeotgali | 60 | 361 | | |
| A0A1A8VMS3 | BS | 22000483450 | Lysine decarboxylase, | catalytic activity | Plasmodium malariae | 41 | 362 | | |
| | | | putative | | | | | | |
| B8KH33 | CG | 22000202167 | Arginine/lysine/ornithine | lysine decarboxylase | gamma proteobacterium NOR5-3 | 134 | 363 | CgCADAV_130 | Cg only |
| | | | decarboxylase | activity | | | | | |
| A0A098FZR5 | CG | 22000202167 | Lysine decarboxylase, | lysine decarboxylase | Legionella fallonii LLAP-10 | 135 | 364 | CgCADAV_121 | Cg only |
| | | | constitutive | activity | | | | | |
| V5AFU2 | BS | 22000483450 | Lysine decarboxylase, | lysine decarboxylase | Betaproteobacteria bacterium | 92 | 365 | | |
| | | | inducible | activity | MOLA814 | | | | |
| A0A1G4H786 | CG | 22000202167 | Lysine decarboxylase, | lysine decarboxylase | Plasmodium vivax | 136 | 366 | | |
| | | | putative | activity | | | | | |
| E8UEY5 | YL | 22000483434 | Arginine, Ornithine and | arginine decarboxylase | Taylorella equigenitalis (strain | 66 | 367 | | |
| | | | Lysine decarboxylase | activity; lysine | MCE9) | | | | |
| | | | | decarboxylase activity; | | | | | |
| | | | | ornithine decarboxylase | | | | | |
| | | | | activity | | | | | |
| A0A067Z2D8 | CG | 22000202167 | Ornithine decarboxylase | lysine decarboxylase | Gluconobacter oxydans DSM 3504 | 137 | 368 | | |
| | | | Ldc | activity; ornithine | | | | | |
| | | | | decarboxylase activity | | | | | |
| A0A101I516 | BS | 22000483450 | Arginine decarboxylase | catalytic activity | candidate division TA06 bacterium | 67 | 369 | | |
| | | | Lysine decarboxylase | | 34_109 | | | | |
| | | | Ornithine decarboxylase | | | | | | |
| A6UJZ3 | CG | 22000202167 | Lysine decarboxylase | lysine decarboxylase | Sinorhizobium medicae (strain | 138 | 370 | CgCADAV_122 | Cg only |
| | | | | activity | WSM419) (Ensifer medicae) | | | | |
| P52095 | CG | 22000202167 | Constitutive lysine | identical protein | Escherichia coli (strain K12) | 44 | 371 | CgCADAV_92, | Cg only |
| | | | decarboxylase | binding; lysine | | | | CgCADAV_123, | |
| | | | | decarboxylase activity | | | | CgCADAV_124, | |
| | | | | | | | | CgCADAV_125, | |
| | | | | | | | | CgCADAV_126, | |
| | | | | | | | | CgCADAV_127 and | |
| | | | | | | | | CgCADAV_128 | |
| A0A1M4RP40 | BS | 22000483450 | Arginine decarboxylase/ | arginine decarboxylase | Staphylococcus aureus | 80 | 372 | BsCADAV_05 | Bs only |
| | | | Lysine decarboxylase | activity | | | | | |
| A0A1D7W8T4 | BS | 22000483450 | Arginine decarboxylase | arginine decarboxylase | Brevibacterium linens | 33 | 373 | | |
| | | | | activity; lysine | | | | | |
| | | | | decarboxylase activity | | | | | |
| W8X0P9 | CG | 22000202167 | Arginine decarboxylase | arginine decarboxylase | Castellaniella defragrans 65Phen | 24 | 374 | CgCADAV_88 and | Cg & Sc |
| | | | Ornithine decarboxylase | activity; lysine | | | | ScCADAV_81 | |
| | | | Lysine decarboxylase | decarboxylase activity; | | | | | |
| | | | | ornithine decarboxylase | | | | | |
| | | | | activity | | | | | |
| P0A9H4 | CG | 22000202167 | Inducible lysine | lysine decarboxylase | Escherichia coli O157:H7 | 11 | 375 | CgCADAV_92 and | Cg only |
| | | | decarboxylase | activity | | | | CgCADAV_73 | |
| A0A0A5GAB3 | BS | 22000483450 | Lysine decarboxylase | catalytic activity | Pontibacillus halophilus JSM | 103 | 376 | | |
| | | | | | 076056 = DSM 19796 | | | | |
| R7HED2 | YL | 22000483434 | Lysine decarboxylase | catalytic activity | Eubacterium sp. CAG:38 | 58 | 377 | | |
| R6FYX1 | YL | 22000483434 | Arginine/lysine | catalytic activity | Clostridium sp. CAG:221 | 22 | 378 | | |
| | | | decarboxylase | | | | | | |
| Q7NFN7 | CG | 22000202167 | Lysine decarboxylase | catalytic activity | Gloeobacter violaceus (strain | 40 | 379 | CgCADAV_93 and | Cg & Sc |
| | | | | | PCC 7421) | | | ScCADAV_85 | |
| A0A0A5GAB3 | CG | 22000202167 | Lysine decarboxylase | catalytic activity | Pontibacillus halophilus JSM | 103 | 380 | | |
| | | | | | 076056 = DSM 19796 | | | | |
| A0A1V0SRU9 | CG | 22000202167 | Lysine decarboxylase | catalytic activity | Sporosarcina ureae | 62 | 381 | CgCADAV_78 | Cg only |
| G8NRB8 | CG | 22000202167 | Lysine decarboxylase | lysine decarboxylase | Granulicella mallensis (strain | 139 | 382 | CgCADAV_129 | Cg only |
| | | | | activity | ATCC BAA-1857/DSM 23137/ | | | | |
| | | | | | MP5ACTX8) | | | | |
| B3PWQ0 | CG | 22000202167 | Probable lysine | lysine decarboxylase | Rhizobium etli (strain CIAT 652) | 50 | 383 | CgCADAV_90 and | Cg & Sc |
| | | | decarboxylase protein | activity | | | | ScCADAV_83 | |
| Q5L130 | YL | 22000483434 | Lysine decarboxylase | lysine decarboxylase | Geobacillus kaustophilus (strain | 119 | 384 | | |
| | | | | activity | HTA426) | | | | |
| Q0I358 | CG | 22000202167 | L-lysine decarboxylase | lysine decarboxylase | Haemophilus somnus (strain 129Pt) | 124 | 385 | | |
| | | | | activity | (Histophilus somni) | | | | |
| A0A1G9YTS7 | YL | 22000483434 | Lysine decarboxylase | catalytic activity | Sediminibacillus halophilus | 13 | 386 | | |
| A0A168T111 | YL | 22000483434 | Lysine decarboxylase | catalytic activity | Phormidium willei BDU 130791 | 100 | 387 | | |
| I2AZB6 | CG | 22000202167 | Lysine decarboxylase | lysine decarboxylase | Francisella noatunensis subsp. | 140 | 388 | CgCADAV_115 | Cg only |
| | | | | activity | orientalis (strain Toba 04) | | | | |
| A0A0A2ARD9 | CG | 22000202167 | Lysine decarboxylase | lysine decarboxylase | Prochlorococcus marinus str. | 90 | 389 | CgCADAV_88 and | Cg & Sc |
| | | | | activity | MIT 9314 | | | ScCADAV_81 | |
| A0A168T111 | BS | 22000483450 | Lysine decarboxylase | catalytic activity | Phormidium willei BDU 130791 | 100 | 390 | | |
| D1Y747 | CG | 22000202167 | Orn/Lys/Arg | lysine decarboxylase | Pyramidobacter piscolens W5455 | 141 | 391 | CgCADAV_130 | Cg only |
| | | | decarboxylase, major | activity | | | | | |
| | | | domain protein | | | | | | |
| A0A1A8VN60 | BS | 22000483450 | Lysine decarboxylase, | catalytic activity | Plasmodium ovale curtisi | 54 | 392 | | |
| | | | putative | | | | | | |
| A0A0U3TRI7 | CG | 22000202167 | Lysine decarboxylase, | lysine decarboxylase | Pseudomonas aeruginosa | 142 | 393 | CgCADAV_108 | Cg only |
| | | | constitutive | activity | | | | | |
| G0V456 | CG | 22000202167 | Arginine decarboxylase | arginine decarboxylase | Caloramator australicus RC3 | 143 | 394 | | |
| | | | Lysine decarboxylase | activity; lysine | | | | | |
| | | | Ornithine decarboxylase | decarboxylase activity; | | | | | |
| | | | | ornithine decarboxylase | | | | | |
| | | | | activity | | | | | |
| W8UMZ9 | CG | 22000202167 | Lysine decarboxylase | lysine decarboxylase | Klebsiella pneumoniae 30684/ | 144 | 395 | CgCADAV_131 | Cg only |
| | | | | activity | NJST258_2 | | | | |
| Q2JVN4 | YL | 22000483434 | Orn/Lys/Arg decarboxylase | catalytic activity | Synechococcus sp. (strain | 70 | 396 | | |
| | | | | | JA-3-3Ab) (Cyanobacteria | | | | |
| | | | | | bacterium Yellowstone A-Prime) | | | | |
| A0A150LIS5 | CG | 22000202167 | Arginine decarboxylase | arginine decarboxylase | Anoxybacillus flavithermus | 79 | 397 | CgCADAV_91 | Cg only |
| | | | | activity; lysine | | | | | |
| | | | | decarboxylase activity | | | | | |
| A0A011QHL8 | CG | 22000202167 | Lysine decarboxylase, | lysine decarboxylase | Candidatus Accumulibacter sp. | 145 | 398 | | |
| | | | constitutive | activity | BA-92 | | | | |
| Q5L130 | CG | 22000202167 | Lysine decarboxylase | lysine decarboxylase | Geobacillus kaustophilus (strain | 119 | 399 | ScCADAV_76 | Sc only |
| | | | | activity | HTA426) | | | | |
| A3CUZ9 | CG | 22000202167 | Lysine decarboxylase | lysine decarboxylase | Methanoculleus marisnigri (strain | 146 | 400 | CgCADAV_99 | Cg only |
| | | | | activity | ATCC 35101/DSM 1498/JR1) | | | | |
| A8I481 | YL | 22000483434 | Lysine decarboxylase | catalytic activity | Chlamydomonas reinhardtii | 82 | 401 | | |
| | | | | | (Chlamydomonas smithii) | | | | |
| A0A1M4T9I0 | YL | 22000483434 | Lysine decarboxylase | catalytic activity | Alkalibacter saccharofermentans | 73 | 402 | | |
| | | | | | DSM 14828 | | | | |
| A8I481 | CG | 22000202167 | Lysine decarboxylase | catalytic activity | Chlamydomonas reinhardtii | 82 | 403 | CgCADAV_87 and | Cg & Sc |
| | | | | | (Chlamydomonas smithii) | | | ScCADAV_80 | |
| A0A1L8CVK5 | YL | 22000483434 | Lysine decarboxylase | catalytic activity | Carboxydothermus pertinax | 115 | 404 | | |
| A0A1K1WST1 | YL | 22000483434 | Lysine decarboxylase | catalytic activity | Thermoactinomyces sp. DSM 45891 | 116 | 405 | | |
| A0A1K1WST1 | CG | 22000202167 | Lysine decarboxylase | catalytic activity | Thermoactinomyces sp. DSM 45891 | 116 | 406 | CgCADAV_85 | Cg only |
| Q9KV75 | CG | 22000202167 | Lysine decarboxylase, | lysine decarboxylase | Vibrio cholerae serotype O1 | 147 | 407 | CgCADAV_132 | Cg only |
| | | | inducible | activity | (strain ATCC 39315/El Tor Inaba | | | | |
| | | | | | N16961) | | | | |
| E8UEY5 | CG | 22000202167 | Arginine, Ornithine and | arginine decarboxylase | Taylorella equigenitalis | 66 | 408 | CgCADAV_90 and | Cg & Sc |
| | | | Lysine decarboxylase | activity; lysine | (strain MCE9) | | | ScCADAV_83 | |
| | | | | decarboxylase activity; | | | | | |
| | | | | ornithine decarboxylase | | | | | |
| | | | | activity | | | | | |
| P48570 | CG | 22000202167 | Homocitrate synthase, | homocitrate synthase | Saccharomyces cerevisiae (strain | 148 | 409 | CgCADAV_92 | Cg only |
| | | | cytosolic isozyme | activity | ATCC 204508/S288c) (Baker's yeast) | | | | |
| A0A0K3BNH0 | CG | 22000202167 | Arginine decarboxylase | arginine decarboxylase | Kibdelosporangium sp. MJ126-NF4 | 149 | 410 | CgCADAV_102 | Cg only |
| | | | | activity; lysine | | | | | |
| | | | | decarboxylase activity; | | | | | |
| | | | | ornithine decarboxylase | | | | | |
| | | | | activity | | | | | |

CG = codon-optimized for Corynebacterim glutamicum; codon-optimized for BS = Bacillus subtilus; codon-optimized for YL = Yarrowia lipolytica. The codon optimizations tested were based on the Kazusa codon usage tables tabulated for each host for gene codon optimization (www.kazusa.or.jp/codon/).

Microbial Host Cells

Any microbe that can be used to express introduced genes can be engineered for fermentative production of 1,5-diaminopentane as described above. In certain embodiments, the microbe is one that is naturally incapable of fermentative production of 1,5-diaminopentane. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T viride, T koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative 1,5-diaminopentane production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum, S. cerevisiae, and B. subtilis cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

Engineered Microbial Cells

The above-described methods can be used to produce engineered microbial cells that produce, and in certain embodiments, overproduce, 1,5-diaminopentane. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for 1,5-diaminopentane production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous lysine decarboxylase, such as in the case of a microbial host cell that does not naturally produce 1,5-diaminopentane. In various embodiments, the microbial cell can include and express, for example: (1) a single heterologous lysine decarboxylase gene, (2) two or more heterologous lysine decarboxylase genes, which can be the same or different (in other words, multiple copies of the same heterologous lysine decarboxylase gene can be introduced or multiple, different heterologous lysine decarboxylase genes can be introduced), (3) a single heterologous lysine decarboxylase gene that is not native to the cell and one or more additional copies of an native lysine decarboxylase gene (if applicable), or (4) two or more non-native lysine decarboxylase genes, which can be the same or different, and one or more additional copies of an native lysine decarboxylase gene (if applicable).

This engineered host cell can include at least one additional genetic alteration that increases flux through the pathway leading to the production of lysine (the immediate precursor of 1,5-diaminopentane). As discussed above, this can be accomplished by one or more of the following: increasing the activity of upstream enzymes, increasing the NaDPH supply, reducing precursor consumption.

In addition, the engineered host cell can express a 1,6-diaminopentane transporter to enhance transport of this compound from inside the engineered microbial cell to the culture medium.

The engineered microbial cells can contain introduced genes that have a native nucleotide sequence or that differ from native. For example, the native nucleotide sequence can be codon-optimized for expression in a particular host cell. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in bacterial cells, namely C. glutamicum and B. subtilis (prokaryotes), and in fungal cells, namely the yeast S. cerevisiae (eukaryotes). (See Example 1.) Other microbial hosts of particular interest include Y. lypolytica.

Illustrative Engineered Bacterial Cells

In certain embodiments, the engineered bacterial (e.g., C. glutamicum) cell expresses one or more heterologous lysine decarboxylase(s) having at least 70 percent, 75 percent, 80 percent, 85 percent, 90 percent, 95 percent or 100 percent amino acid sequence identity with a lysine decarboxylase from Escherichia coli (strain K12), Escherichia coli O157:H7, Vibrio cholerae serotype 01 (strain ATCC39315/El Tor Inaba N16961), Escherichia coli MS 117-3, Candidatus Burkholderia crenata, and/or butyrate-producing bacterium SS3/4. In particular embodiments:

the Escherichia coli (strain K12) lysine decarboxylase includes SEQ ID NO:44;

the Escherichia coli O157:H7 lysine decarboxylase includes SEQ ID NO:11;

the Vibrio cholerae serotype 01 (strain ATCC39315/El Tor Inaba N16961) lysine decarboxylase includes SEQ ID NO:147;

the Escherichia coli MS 117-3 lysine decarboxylase includes SEQ ID NO:87;

the Candidatus Burkholderia crenata lysine decarboxylase includes SEQ ID NO:97; and

the butyrate-producing bacterium SS3/4 lysine decarboxylase includes SEQ ID NO:30. As noted above, a titer of about 5.5 gm/L was achieved in C. glutamicum by expressing lysine decarboxylases from each of Escherichia coli MS 117-3, Candidatus Burkholderia crenata, and butyrate-producing bacterium SS3/4. (CgCADAV_107, expressing SEQ ID NOs:87, 97, and 30; see Table 5). A titer of about 7.0 gm/L was achieved by additionally expressing a lysine decarboxylase from a mine drainage metagenome (SEQ ID NO:93), together with these enzymes.

In certain embodiments, the engineered bacterial (e.g., B. subtilis) cell expresses one or more heterologous lysine decarboxylase(s) having at least 70 percent, 75 percent, 80 percent, 85 percent, 90 percent, 95 percent or 100 percent amino acid sequence identity with a lysine decarboxylase from Clostridium CAG:221, Clostridium CAG:288, and/or Staphylococcus aureus. In particular embodiments:

the Clostridium CAG:221 lysine decarboxylase includes SEQ ID NO:22;

the Clostridium CAG:288 lysine decarboxylase includes SEQ ID NO:15; and

the Staphylococcus aureus lysine decarboxylase includes SEQ ID NO:80. As noted above, a titer of about 47 mg/L was achieved in B. subtilis by expressing lysine decarboxylases from each of Clostridium CAG:221, Clostridium CAG:288, and Staphylococcus aureus. (See FIG. 4.)

Illustrative Engineered Yeast Cells

In certain embodiments, the engineered yeast (e.g., S. cerevisiae) cell expresses a heterologous (e.g., non-native) lysine decarboxylase having at least 70 percent, 75 percent, 80 percent, 85 percent, 90 percent, 95 percent, or 100 percent amino acid sequence identity to a lysine decarboxylase from Yersinia enterocolitica W22703, Castellaniella detragans 65Phen, and/or Prochorococcus marinus str. IT 9314. In particular embodiments:

the Yersinia enterocolitica W22703 lysine decarboxylase includes SEQ ID NO:6;

the Castellaniella detragans 65Phen lysine decarboxylase includes SEQ ID NO:24; and

the Prochorococcus marinus str. IT 9314 includes SEQ ID NO:90. As noted above, a titer of about 5 mg/L was achieved in S. cerevisiae by expressing lysine decarboxylases from each of Yersinia enterocolitica W22703, Castellaniella detragans 65Phen, and/or Prochorococcus marinus str. IT 9314. (See FIG. 3.)

These may be the only genetic alterations of the engineered yeast cell, or the yeast cell can include one or more additional genetic alterations, as discussed more generally above.

Culturing of Engineered Microbial Cells

Any of the microbial cells described herein can be cultured, e.g., for maintenance, growth, and/or 1,5-diaminopentane production.

In some embodiments, the cultures are grown to an optical density at 600 nm of 10-500, such as an optical density of 50-150.

In various embodiments, the cultures include produced 1,5-diaminopentane at titers of at least 10, 25, 50, 75, 100, 200, 300, 400, 500, 600, 700, 800, or 900 μg/L, or at least 1, 10, 50, 75, 100, 200, 300, 400, 500, 600, 700, 800, or 900 mg/L or at least 1, 1.5, 2, 2.5, 3, 3.5, 4, 4.5, 5, 10, 20, 50 g/L. In various embodiments, the titer is in the range of 10 μg/L to 10 g/L, 25 μg/L to 20 g/L, 100 μg/L to 10 g/L, 200 μg/L to 5 g/L, 500 μg/L to 4 g/L, 1 mg/L to 3 g/L, 500 mg/L to 2 g/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce 1,5-diaminopentane, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

Illustrative materials and methods suitable for the maintenance and growth of the engineered microbial cells described herein can be found below in Example 1.

1,5-Diaminopentane Production and Recovery

Any of the methods described herein may further include a step of recovering 1,5-diaminopentane. In some embodiments, the produced 1,5-diaminopentane contained in a so-called harvest stream is recovered/harvested from the production vessel. The harvest stream may include, for instance, cell-free or cell-containing aqueous solution coming from the production vessel, which contains 1,5-diaminopentane as a result of the conversion of production substrate by the resting cells in the production vessel. Cells still present in the harvest stream may be separated from the 1,5-diaminopentane by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced 1,5-diaminopentane from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify 1,5-diaminopentane. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—Construction and Selection of Strains of Corynebacteria glutamicum, Saccharomyces Cerevisiae, and Bacillus subtilis Engineered to Produce 1,5-Diaminopentane

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum and B. subtilis Pathway Integration

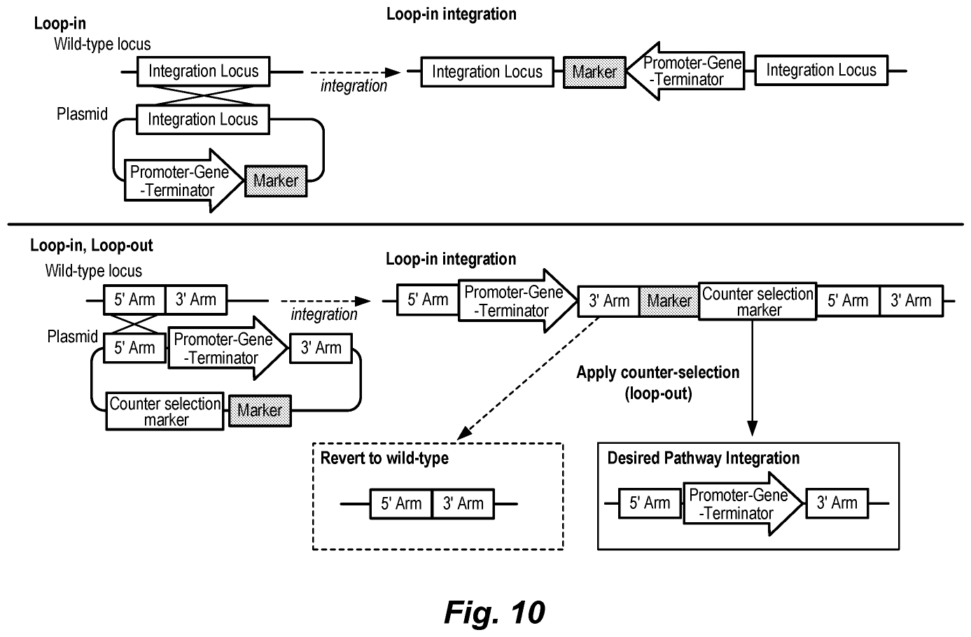

A “loop-in, single-crossover” genomic integration strategy has been developed to engineer C. glutamicum and B. subtilis strains. FIG. 10 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum or B. subtilis chromosome. Integration events are stably maintained in the genome by growth in the presence of antibiotic (25 μg/mlkanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

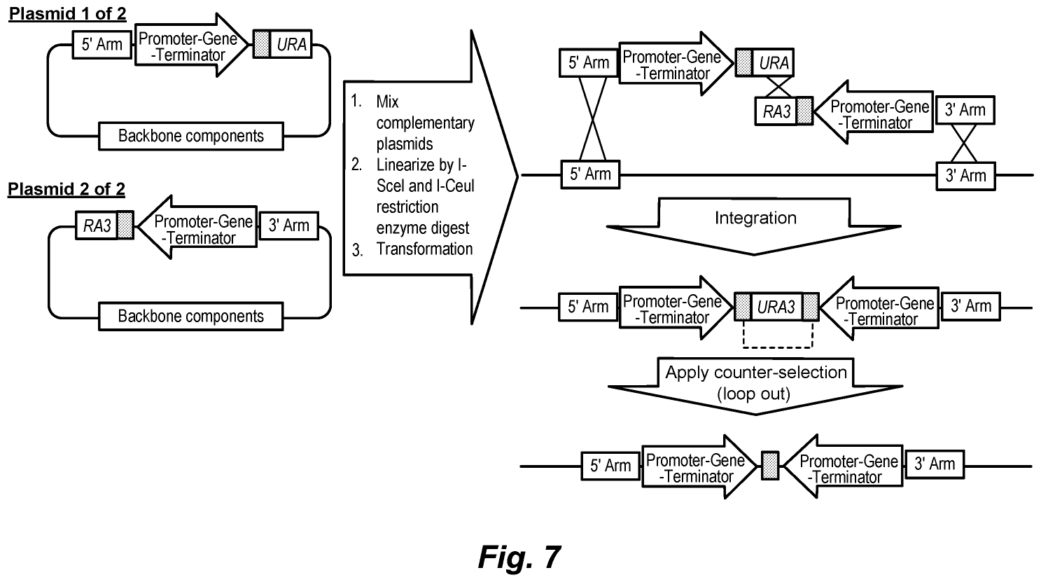

A “split-marker, double-crossover” genomic integration strategy has been developed to engineer S. cerevisiae strains. FIG. 7 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for S. cerevisiae involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (SD-ura for S. cerevisiae) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

First-Round Genetic Engineering Results in Corynebacteria glutamicum, Saccharomyces cerevisiae, and Bacillus subtilis

A library approach was taken to screen heterologous pathway enzymes to establish the 1,5-diaminopentane pathway. The lysine decarboxylases tested were codon-optimized as shown in the SEQ ID NO Cross-Reference Table above and expressed in Corynebacteria glutamicum, Saccharomyces cerevisiae, and Bacillus subtilis hosts.

First-round genetic engineering results are shown in FIGS. 2 (C. glutamicum), 3 (S. cerevisiae), and 4 (B. subtilis). In C. glutamicum, a 300 mg/L titer of 1,5-diaminopentane was achieved in a first round of engineering after integration of three lysine decarboxylases from Escherichia coli (strain K12), Escherichia coli O157:H7, and Vibrio cholerae serotype 01 (strain ATCC39315/El Tor Inaba N16961; SEQ ID NOs:44, 11, and 147, respectively). (See FIG. 2.)

In S. cerevisiae, a titer of 5 mg/L was achieved in a first round of engineering after integration of three lysine decarboxylases from Yersinia enterocolitica W22703, Castellaniella detragans 65Phen, and Prochorococcus marinus str. IT 9314 (; SEQ ID NOs:6, 24, and 90, respectively). (See FIG. 3.)

In B. subtilis, a titer of about 47 mg/L was achieved in a first round of engineering after integration of lysine decarboxylases from each of Clostridium CAG:221, Clostridium CAG:288, and Staphylococcus aureus (; SEQ ID NOs:22, 15, and 80, respectively). (See FIG. 4.)

Second-Round Genetic Engineering Results in Corynebacteria glutamicum

A second round of engineering was carried out in the C. glutamicum. A titer of about 5.5 gm/L was achieved after integration of lysine decarboxylases from each of Escherichia coli MS 117-3, Candidatus Burkholderia crenata, and butyrate-producing bacterium SS3/4 (SEQ ID NOs:87, 97, and 30, respectively). (See FIG. 5).

Third-Round Genetic Engineering Results in Corynebacteriaglutamicum

A second round of engineering was carried out in the C. glutamicum. A titer of about 7.0 gm/L was achieved after insertion of an additional lysine decarboxylase from a mine drainage metagenome (SEQ ID NO:93) into the best-producing strain from the second-round (CgCADAV_107, including SEQ ID NOs:87, 97, and 30). See CgCADAV_306 in FIG. 11).

### Example 2—Bioreactor Production Run of Corynebacteria glutamicum Engineered to Produce 1,5-diaminopentane

An engineered C. glutamicum strain (CgCADAV_107) expressing lysine decarboxylases from each of Escherichia coli MS 117-3, Candidatus Burkholderia crenata, and butyrate-producing bacterium SS3/4 (SEQ ID NOs:87, 97, and 30, respectively) was tested for 1,5-diaminopentane production in bioreactor production runs.

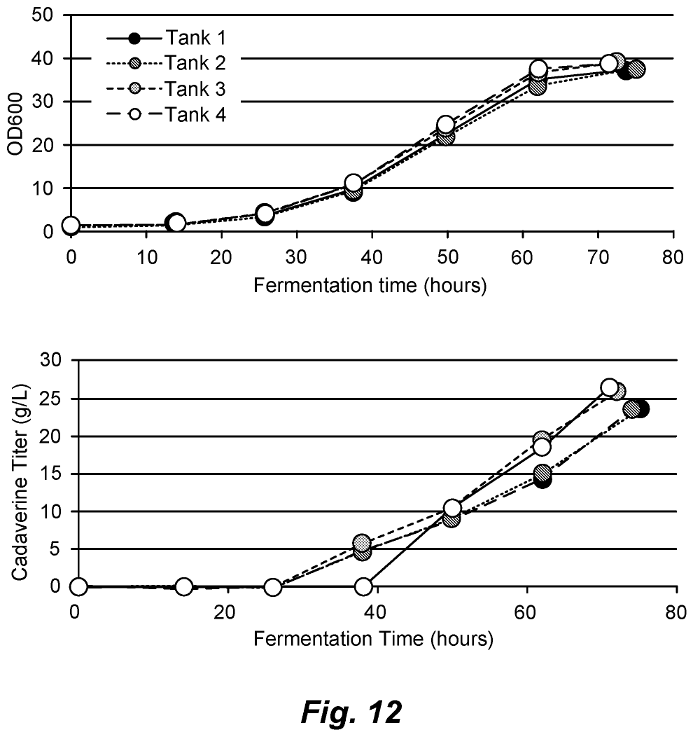

As indicated in FIG. 12, bioreactor production runs using CgCADAV_107 resulted in 1,5-diaminopentane titers of about 27 gm/L.

What is claimed is:

1. An engineered microbial cell that expresses a non-native lysine decarboxylase, wherein the engineered microbial cell produces 1,5-diaminopentane.

2. The engineered microbial cell of claim 1, wherein the engineered microbial cell also expresses a non-native 1,5-diaminopentane transporter.

3. The engineered microbial cell of claim 1 or claim 2, wherein the engineered microbial cell expresses one or more additional enzyme(s) selected from an additional non-native lysine decarboxylase and/or an additional non-native 1,5-diaminopentane transporter.

4. The engineered microbial cell of claim 3, wherein the additional enzyme(s) are from a different organism than the corresponding enzyme in claim 1 or claim 2.

5. The engineered microbial cell of claim 3 or claim 4, wherein the additional enzyme(s) comprise(s) one or more additional copies of the corresponding enzyme in claim 1 or claim 2.

6. The engineered microbial cell of any of claims 1-5, wherein the engineered microbial cell comprises increased activity of one or more upstream lysine pathway enzyme(s), said increased activity being increased relative to a control cell.

7. The engineered microbial cell of any of claims 1-6, wherein the engineered microbial cell comprises increased activity of one or more enzyme(s) that increase the supply of the reduced form of nicotinamide adenine dinucleotide phosphate (NADPH), said increased activity being increased relative to a control cell.

8. The engineered microbial cell of claim 7, wherein the one or more enzyme(s) that increase the supply of the reduced form of NADPH is selected from the group consisting of pentose phosphate pathway enzymes, NADP+-dependent glyceraldehyde 3-phosphate dehydrogenase (GAPDH), and NADP+-dependent glutamate dehydrogenase.

9. The engineered microbial cell of any one of claims 1-8, wherein the engineered microbial cell comprises reduced activity of one or more enzyme(s) that consume one or more lysine pathway precursors, said reduced activity being reduced relative to a control cell.

10. The engineered microbial cell of any one of claims 1-9, wherein the engineered microbial cell comprises reduced activity of a native lysine exporter, said reduced activity being reduced relative to a control cell.

11. The engineered microbial cell of claim 10, wherein the native lysine exporter is Corynebacterium glutamicum lysE or an ortholog thereof.

12. The engineered microbial cell of any one of claims 1-11, wherein the engineered microbial cell comprises reduced expression of the C. glutamicum NCg10561 gene or an ortholog thereof, said reduced expression being reduced relative to a control cell.

13. The engineered microbial cell of any one of claims 1-12, wherein the engineered microbial cell comprises reduced expression of the C. glutamicum trpB gene or an ortholog thereof, said reduced expression being reduced relative to a control cell.

14. The engineered microbial cell of any one of claims 9-13, wherein the reduced activity is achieved by one or more means selected from the group consisting of gene deletion, gene disruption, altering regulation of a gene, and replacing a native promoter with a less active promoter.

15. An engineered microbial cell, wherein the engineered microbial cell comprises means for expressing a non-native lysine decarboxylase, and wherein the engineered microbial cell produces 1,5-diaminopentane.

16. The engineered microbial cell of claim 15, wherein the engineered microbial cell also comprises means for expressing a non-native 1,5-diaminopentane transporter.

17. The engineered microbial cell of claim 15 or claim 16, wherein the engineered microbial cell means for expressing one or more additional enzyme(s) selected from an additional non-native lysine decarboxylase and/or an additional non-native 1,5-diaminopentane transporter.

18. The engineered microbial cell of claim 17, wherein the additional enzyme(s) are from a different organism than the corresponding enzyme in claim 15 or claim 16.

19. The engineered microbial cell of any of claims 15-18 wherein the engineered microbial cell comprises means for increasing activity of one or more upstream lysine pathway enzyme(s), said activity being increased relative to a control cell.

20. The engineered microbial cell of any of claims 15-19, wherein the engineered microbial cell comprises means for increasing activity of one or more enzyme(s) that increase the NADPH supply, said activity being increased relative to a control cell.

21. The engineered microbial cell of claim 20, wherein the one or more enzyme(s) that increase the supply of the reduced form of nicotinamide adenine dinucleotide phosphate (NADPH) is selected from the group consisting of pentose phosphate pathway enzymes, NADP+-dependent glyceraldehyde 3-phosphate dehydrogenase (GAPDH), and NADP+-dependent glutamate dehydrogenase.

22. The engineered microbial cell of any one of claims 15-21, wherein the engineered microbial cell comprises means for reducing activity of one or more enzyme(s) that consume one or more lysine pathway precursors, said activity being reduced relative to a control cell.

23. The engineered microbial cell of any one of claims 15-22, wherein the engineered microbial cell comprises means for reducing activity of a native lysine exporter, said activity being reduced relative to a control cell.

24. The engineered microbial cell of claim 23, wherein the native lysine exporter is Corynebacterium glutamicum lysE or an ortholog thereof.

25. The engineered microbial cell of any one of claims 15-24, wherein the engineered microbial cell comprises means for reducing expression of the C. glutamicum NCg10561 gene or an ortholog thereof, said expression being reduced relative to a control cell.

26. The engineered microbial cell of any one of claims 15-25, wherein the engineered microbial cell comprises means for reducing expression of the C. glutamicum trpB gene or an ortholog thereof, said expression being reduced relative to a control cell.

27. The engineered microbial cell of any one of claims 1-26, wherein the engineered microbial cell is a bacterial cell.

28. The engineered microbial cell of claim 27, wherein the bacterial cell is a cell of the genus Corynebacteria.

29. The engineered microbial cell of claim 28, wherein the bacterial cell is a cell of the species glutamicum.

30. The engineered microbial cell of claim 29, wherein the non-native lysine decarboxylase comprises a lysine decarboxylase having at least 70% amino acid sequence identity with a lysine decarboxylase selected from the group consisting of Escherichia coli, Vibrio cholerae, Candidatus Burkholderia crenata, butyrate-producing bacterium, and any combination thereof.

31. The engineered microbial cell of claim 30, wherein the cell comprises at least three different lysine decarboxylases.

32. The engineered microbial cell of claim 31, wherein the engineered microbial cell comprises three non-native lysine decarboxylases having at least 70% amino acid sequence identity with each of the lysine decarboxylases from Escherichia coli, Candidatus Burkholderia crenata, and butyrate-producing bacterium.

33. The engineered microbial cell of claim 32, wherein the engineered microbial cell additionally comprises a non-native lysine decarboxylase having at least 70% amino acid sequence identity with a lysine decarboxylase from a mine drainage metagenome.

34. The engineered microbial cell of claim 33, wherein the lysine decarboxylases from Escherichia coli, Candidatus Burkholderia crenata, butyrate-producing bacterium, and the mine drainage metagenome comprise SEQ ID NOs:87, 97, 30, and 93.

35. The engineered microbial cell of claim 27, wherein the bacterial cell is a cell of the genus Bacillus.

36. The engineered microbial cell of claim 35, wherein the bacterial cell is a cell of the species subtilis.

37. The engineered microbial cell of claim 36, wherein the non-native lysine decarboxylase comprises a lysine decarboxylase having at least 70% amino acid sequence identity with a lysine decarboxylase selected from the group consisting of a Clostridium species, Staphylococcus aureus, and any combination thereof.

38. The engineered microbial cell of claim 37, wherein the cell comprises at least three different lysine decarboxylases.

39. The engineered microbial cell of claim 38, wherein the engineered microbial cell comprises three non-native lysine decarboxylases having at least 70% amino acid sequence identity with each of the lysine decarboxylases from Clostridium CAG:221, Clostridium CAG:288, and Staphylococcus aureus.

40. The engineered microbial cell of any one of claims 1-26, wherein the engineered microbial cell comprises a fungal cell.

41. The engineered microbial cell of claim 40, wherein the engineered microbial cell comprises a yeast cell.

42. The engineered microbial cell of claim 41, wherein the yeast cell is a cell of the genus Saccharomyces.

43. The engineered microbial cell of claim 42, wherein the yeast cell is a cell of the species cerevisiae.

44. The engineered microbial cell of any one of claims 1-43, wherein the non-native lysine decarboxylase comprises a lysine decarboxylase having at least 70% amino acid sequence identity with a lysine decarboxylase selected from the group consisting of Yersinia enterocolitica, Castellaniella detragans, Prochorococcus marinus, and any combination thereof.

45. The engineered microbial cell of claim 44, wherein the cell comprises at least three different lysine decarboxylases.

46. The engineered microbial cell of claim 45, wherein the engineered microbial cell comprises three non-native lysine decarboxylases having at least 70% amino acid sequence identity with each of the lysine decarboxylases from Yersinia enterocolitica, Castellaniella detragans, and Prochorococcus marinus.

47. The engineered microbial cell of any one of claims 1-46, wherein, when cultured, the engineered microbial cell produces 1,5-diaminopentane at a level at least 5 mg/L of culture medium.

48. The engineered microbial cell of claim 47, wherein, when cultured, the engineered microbial cell produces 1,5-diaminopentane at a level at least 5 gm/L of culture medium.

49. The engineered microbial cell of claim 48, wherein, when cultured, the engineered microbial cell produces 1,5-diaminopentane at a level at least 25 gm/L of culture medium.

50. A method of culturing engineered microbial cells according to any one of claims 1-49, the method comprising culturing the cells under conditions suitable for producing 1,5-diaminopentane.

51. The method of claim 50, wherein the method comprises fed-batch culture, with an initial glucose level in the range of 1-100 g/L, followed controlled sugar feeding.

52. The method of claim 50 or claim 51, wherein the fermentation substrate comprises glucose and a nitrogen source selected from the group consisting of urea, an ammonium salt, ammonia, and any combination thereof.

53. The method of any one of claims 50-52, wherein the culture is pH-controlled during culturing.

54. The method of any one of claims 50-53, wherein the culture is aerated during culturing.

55. The method of any one of claims 50-54, wherein the engineered microbial cells produce 1,5-diaminopentane at a level at least 5 mg/L of culture medium.

56. The method of any one of claims 50-55, wherein the method additionally comprises recovering 1,5-diaminopentane from the culture.

57. A method for preparing 1,5-diaminopentane using microbial cells engineered to produce 1,5-diaminopentane, the method comprising:
(a) expressing a non-native lysine decarboxylase in microbial cells;
(b) cultivating the microbial cells in a suitable culture medium under conditions that permit the microbial cells to produce 1,5-diaminopentane, wherein the 1,5-diaminopentane is released into the culture medium; and
(c) isolating 1,5-diaminopentane from the culture medium.
